SEQUENCE LISTING (59 sequences)

SEQ ID NO: 1 - DNA, 90 bp, synthetic construct
  misc_feature 1..90: note = Synthetic probe
  source 1..90: mol_type = other DNA
tttttttttttttttttttttttttttttttttttttttttttttttttttttttttttcgcttcaggtagtagtacgtctatgtatgat

SEQ ID NO: 2 - DNA, 90 bp, synthetic construct
  misc_feature 1..90: note = Synthetic probe
  source 1..90: mol_type = other DNA
atcctgtagcattaatactctcgcagcacaacggtacttttttttttttttttttttttttttttttttttttttttttttttttttttt

SEQ ID NO: 3 - DNA, 18 bp, synthetic construct
  misc_feature 1..18: note = Synthetic connector
  source 1..18: mol_type = other DNA
ctacaggatatcatacat

SEQ ID NO: 4 - DNA, 22 bp, synthetic construct
  misc_feature 1..22: note = Synthetic primer
  source 1..22: mol_type = other DNA
cgcttcaggtagtagtacgtct

SEQ ID NO: 5 - DNA, 22 bp, synthetic construct
  misc_feature 1..22: note = Synthetic primer
  source 1..22: mol_type = other DNA
ccgttgtgctgcgagagtatta

SEQ ID NO: 6 - DNA, 45 bp, synthetic construct
  misc_feature 1..45: note = Synthetic nucleic acid
  source 1..45: mol_type = other DNA
caacgtgtgctcttccgatcttcagctgatcgaatgagtatgcct

SEQ ID NO: 7 - DNA, 30 bp, synthetic construct
  misc_feature 1..30: note = Synthetic nucleic acid
  source 1..30: mol_type = other DNA
gcttactcatacggaacggagctagtaccc

SEQ ID NO: 8 - DNA, 30 bp, synthetic construct
  misc_feature 1..30: note = Synthetic nucleic acid
  source 1..30: mol_type = other DNA
acggagctagtacccgttcatacgtcgcgc

SEQ ID NO: 9 - DNA, 30 bp, synthetic construct
  misc_feature 1..30: note = Synthetic nucleic acid
  source 1..30: mol_type = other DNA
cgaatgagtatgccttgcctcgatcatggg

SEQ ID NO: 10 - DNA, 30 bp, synthetic construct
  misc_feature 1..30: note = Synthetic nucleic acid
  source 1..30: mol_type = other DNA
gttcatacgtcgcgcaagtatctcggacta

SEQ ID NO: 11 - DNA, 30 bp, synthetic construct
  misc_feature 1..30: note = Synthetic nucleic acid
  source 1..30: mol_type = other DNA
aagtatctcggactatcagcatgtagtacg

SEQ ID NO: 12 - DNA, 30 bp, synthetic construct
  misc_feature 1..30: note = Synthetic nucleic acid
  source 1..30: mol_type = other DNA
caagtatgcagcgcgttcatagagcctgat

SEQ ID NO: 13 - DNA, 30 bp, synthetic construct
  misc_feature 1..30: note = Synthetic nucleic acid
  source 1..30: mol_type = other DNA
tcagcatgtagtacgagatcagtgacagtg

SEQ ID NO: 14 - DNA, 47 bp, synthetic construct
  misc_feature 1..47: note = Synthetic nucleic acid
  source 1..47: mol_type = other DNA
agatcaggacagtggtataccagttgagacgcaactatggtgacgaa

SEQ ID NO: 15 - DNA, 30 bp, synthetic construct
  misc_feature 1..30: note = Synthetic nucleic acid
  source 1..30: mol_type = other DNA
agtcgtacatcatgctctagtcactgtcac

SEQ ID NO: 16 - DNA, 22 bp, synthetic construct
  misc_feature 1..22: note = Synthetic nucleic acid
  source 1..22: mol_type = other DNA
cagacgtgtgctcttccgatct

SEQ ID NO: 17 - DNA, 23 bp, synthetic construct
  misc_feature 1..23: note = Synthetic nucleic acid
  source 1..23: mol_type = other DNA
ttcgtcaccatagttgcgtctca

SEQ ID NO: 18 - DNA, 152 bp, synthetic construct
  misc_feature 1..152: note = Synthetic nucleic acid
  source 1..152: mol_type = other DNA
gtctgcacacgagaaggctagaagtcgactagcttactcatacggaacgagctagtacccgttcatacgtcgcgcaagtatctcggactatcagcatgtagtacgagatcagtgacagggtataccagttgagacgcaactatggtgacgaa

SEQ ID NO: 19 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnaacaacaacccaagtatgcagcgcg

SEQ ID NO: 20 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnaactctcgcccaagtatgcagcgcg

SEQ ID NO: 21 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnacagttatgccaagtatgcagcgcg

SEQ ID NO: 22 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnagctacgatgcaagtatgcagcgcg

SEQ ID NO: 23 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnataagagcaccaagtatgcagcgcg

SEQ ID NO: 24 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnccactcgaaccaagtatgcagcgcg

SEQ ID NO: 25 - DNA, 44 bp, synthetic construct
  misc_feature 1..44: note = Synthetic nucleic acid
  variation 16..20: note = n is a, c, g, or t
  source 1..44: mol_type = other DNA
tgcctcgatcatgggnnnnncgagaagaacaagtatgcagcgcg

SEQ ID NO: 26 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnnctctatacaccaagtatgcagcgcg

SEQ ID NO: 27 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnngtagaatcctcaagtatgcagcgcg

SEQ ID NO: 28 - DNA, 45 bp, synthetic construct
  misc_feature 1..45: note = Synthetic nucleic acid
  variation 16..20: note = n is a, c, g, or t
  source 1..45: mol_type = other DNA
tgcctcgatcatgggnnnnntccttaatcccaagtatgcagcgcg

SEQ ID NO: 29 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnnaacaaggtggagtcgtacatcatgc

SEQ ID NO: 30 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnnaagactgagaagtcgtacatcatgc

SEQ ID NO: 31 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnnaccgcaagacagtcgtacatcatgc

SEQ ID NO: 32 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnnaggaattgccagtcgtacatcatgc

SEQ ID NO: 33 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnnatcttggagtagtcgtacatcatgc

SEQ ID NO: 34 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnnccggtagttcagtcgtacatcatgc

SEQ ID NO: 35 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnncggacacctaagtcgtacatcatgc

SEQ ID NO: 36 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnngaggttcagcagtcgtacatcatgc

SEQ ID NO: 37 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnngtccacagctagtcgtacatcatgc

SEQ ID NO: 38 - DNA, 45 bp, synthetic construct
  misc_feature 1..45: note = Synthetic nucleic acid
  variation 16..20: note = n is a, c, g, or t
  source 1..45: mol_type = other DNA
ttcatagagcctgatnnnnntggtgcataaagtcgtacatcatgc

SEQ ID NO: 39 - DNA, 48 bp, synthetic construct
  misc_feature 1..48: note = Synthetic nucleic acid
  source 1..48: mol_type = other DNA
tctagtcactgtcacaaccgattccactctgcgttgataccactgctt

SEQ ID NO: 40 - DNA, 48 bp, synthetic construct
  misc_feature 1..48: note = Synthetic nucleic acid
  source 1..48: mol_type = other DNA
tctagtcactgtcacaatggtaacgactctgcgttgataccactgctt

SEQ ID NO: 41 - DNA, 48 bp, synthetic construct
  misc_feature 1..48: note = Synthetic nucleic acid
  source 1..48: mol_type = other DNA
tctagtcactgtcacacgcctcttaactctgcgttgataccactgctt

SEQ ID NO: 42 - DNA, 48 bp, synthetic construct
  misc_feature 1..48: note = Synthetic nucleic acid
  source 1..48: mol_type = other DNA
tctagtcactgtcacagtgtggtccactctgcgttgataccactgctt

SEQ ID NO: 43 - DNA, 48 bp, synthetic construct
  misc_feature 1..48: note = Synthetic nucleic acid
  source 1..48: mol_type = other DNA
tctagtcactgtcaccaatacgtccactctgcgttgataccactgctt

SEQ ID NO: 44 - DNA, 48 bp, synthetic construct
  misc_feature 1..48: note = Synthetic nucleic acid
  source 1..48: mol_type = other DNA
tctagtcactgtcaccctcattgtcactctgcgttgataccactgctt

SEQ ID NO: 45 - DNA, 48 bp, synthetic construct
  misc_feature 1..48: note = Synthetic nucleic acid
  source 1..48: mol_type = other DNA
tctagtcactgtcacctagcgcgttactctgcgttgataccactgctt

SEQ ID NO: 46 - DNA, 48 bp, synthetic construct
  misc_feature 1..48: note = Synthetic nucleic acid
  source 1..48: mol_type = other DNA
tctagtcactgtcacgcgtcgtgaaactctgcgttgataccactgctt

SEQ ID NO: 47 - DNA, 48 bp, synthetic construct
  misc_feature 1..48: note = Synthetic nucleic acid
  source 1..48: mol_type = other DNA
tctagtcactgtcaccttactcggtactctgcgttgataccactgctt

SEQ ID NO: 48 - DNA, 48 bp, synthetic construct
  misc_feature 1..48: note = Synthetic nucleic acid
  source 1..48: mol_type = other DNA
tctagtcactgtcacttcgatgcggactctgcgttgataccactgctt

SEQ ID NO: 49 - protein, 8 aa, synthetic construct
  REGION 1..8: note = Synthetic peptide
RTRFTGRY

SEQ ID NO: 50 - protein, 13 aa, synthetic construct
  REGION 1..13: note = Synthetic peptide
FVNQHLCGSHLVD

SEQ ID NO: 51 - protein, 7 aa, synthetic construct
  REGION 1..7: note = Synthetic peptide
KCDYWEC

SEQ ID NO: 52 - DNA, 144 bp, synthetic construct
  misc_feature 1..144: note = Synthetic nucleic acid
  source 1..144: mol_type = other DNA
cagacgtgtgctcttccgatcttcagctgatcgaatgagtatgccttgcctcgatcatgggcaagtatgcagcgcgttcatagagcctgatagtcgtacatcatgctctagtcactgtcacactctgcgttgataccactgctt

SEQ ID NO: 53 - DNA, 164 bp, synthetic construct
  misc_feature 1..164: note = Synthetic nucleic acid
  variation 1..14: note = n is a, c, g, or t
  variation 16..20: note = n is a, c, g, or t
  variation 30: note = n is a, c, g, or t
  variation 40..43: note = n is a, c, g, or t
  variation 45: note = n is a, c, g, or t
  variation 48..50: note = n is a, c, g, or t
  variation 82: note = n is a, c, g, or t
  variation 85: note = n is a, c, g, or t
  variation 88: note = n is a, c, g, or t
  variation 91: note = n is a, c, g, or t
  variation 93: note = n is a, c, g, or t
  variation 95..96: note = n is a, c, g, or t
  variation 128..136: note = n is a, c, g, or t
  variation 156..157: note = n is a, c, g, or t
  variation 160..162: note = n is a, c, g, or t
  variation 164: note = n is a, c, g, or t
  source 1..164: mol_type = other DNA
nnnnnnnnnnnnnnannnnncttgcctcgntcatgggtgnnnnanaannnccaagtatgcagcgcgttcatagagcctgatnggnganganangnnagtcgtacatcatgctctagtcactgtcaccnnnnnnnnnactctgcgttgataccactnnttnnnan

SEQ ID NO: 54 - DNA, 152 bp, synthetic construct
  misc_feature 1..152: note = Synthetic nucleic acid
  source 1..152: mol_type = other DNA
aagcagtggtatcaacgcagagtgtgacagtgactagagcatgatgtacgactatcaggctctatgaacgcgctgcatacttgcccatgatcgaggcaaggcatactcattcgatcagctgaagatcggaagatcggaagagcacacgtctg

SEQ ID NO: 55 - DNA, 158 bp, synthetic construct
  misc_feature 1..158: note = Synthetic nucleic acid
  variation 1..14: note = n is a, c, g, or t
  variation 16..17: note = n is a, c, g, or t
  variation 24: note = n is a, c, g, or t
  variation 38..39: note = n is a, c, g, or t
  variation 42..43: note = n is a, c, g, or t
  variation 45..48: note = n is a, c, g, or t
  variation 50..52: note = n is a, c, g, or t
  variation 84..88: note = n is a, c, g, or t
  variation 92: note = n is a, c, g, or t
  variation 95..97: note = n is a, c, g, or t
  variation 158: note = n is a, c, g, or t
  source 1..158: mol_type = other DNA
nnnnnnnnnnnnnngnntagagcntgatgtacgactgnngtnncnnnncnnnatcaggctctatgaacgcgctgcatacttggnnnnnctgnccnnncccatgatcgaggcaaggcatactcattcgatcagctgaagatcggaagagcacacgctgn

SEQ ID NO: 56 - DNA, 250 bp, synthetic construct
  misc_feature 1..250: note = Synthetic nucleic acid
  source 1..250: mol_type = other DNA
cagacgtgtgctcttccgatcttcagctgatcgaatgagtatgccttgcctcgatcatgggcctgcagctaccatgcagtatgcagcgcgttcatagagcctgatgctgtgtccacagctagtcgtacatcatgctctagtcactgtcacaatggtaacgactctgcgttgataccactgcttagatcggaagagcacacgtctgaactccagtcacccaacattatctcgtatgccgtcttctgcttga

SEQ ID NO: 57 - DNA, 138 bp, synthetic construct
  misc_feature 1..138: note = Synthetic nucleic acid
  variation 16..20: note = n is a, c, g, or t
  variation 61..65: note = n is a, c, g, or t
  source 1..138: mol_type = other DNA
tgcctcgatcatgggnnnnnagctaccatgcaagtatgcagcgcgttcatagagcctgatnnnnngtccacagctagtcgtacatcatgctctagtcactgtcacaatggtaacgactctgcgttgataccactgctt

SEQ ID NO: 58 - DNA, 250 bp, synthetic construct
  misc_feature 1..250: note = Synthetic nucleic acid
  source 1..250: mol_type = other DNA
caagcagaagacggcatacgagataatgttgggtgactggagttcagacgtgtgctcttccgatctcagacgtgtgctcttccgatcttcagctgatcgaatgagtatgccttgcctcgatcatgggtgaagagagacggagcaagtatgcagcgcgttcatagagcctgatgctgtgtcccccgcaagtcgtacatcatgctctagtcactgtcacaatggtaacgactctgcgttgataccactgctt

SEQ ID NO: 59 - DNA, 138 bp, synthetic construct; identical to SEQ ID NO: 57
  misc_feature 1..138: note = Synthetic nucleic acid
  variation 16..20: note = n is a, c, g, or t
  variation 61..65: note = n is a, c, g, or t
  source 1..138: mol_type = other DNA

(19) United States
(12) Patent Application Publication
(10) Pub. No.: US 2025/0164498 A1
(43) Pub. Date: May 22, 2025

(54) MOLECULAR NEIGHBORHOOD DETECTION BY OLIGONUCLEOTIDES

(71) Applicant: Board of Regents, The University of Texas System, Austin, TX (US)

(72) Inventors: Edward MARCOTTE, Austin, TX (US); Jagannath SWAMINATHAN, Austin, TX (US); Andrew ELLINGTON, Austin, TX (US); Alexander BOULGAKOV, Austin, TX (US); Jon LAURENT, Austin, TX (US); Raghav SHROFF, Austin, TX (US); Erhu XIONG, Austin, TX (US); Sanchita BHADRA, Austin, TX (US); Brendan FLOYD, Austin, TX (US); Eric ANSLYN, Austin, TX (US)

(73) Assignee: Board of Regents, The University of Texas System, Austin, TX (US)

(21) Appl. No.: 18/968,147

(22) Filed: Dec. 4, 2024

Related U.S. Application Data
(63) Continuation of application No. 17/146,165, filed on Jan. 11, 2021, now Pat. No. 12,196,760.
Application No. 17/146,165 is a continuation of application No. PCT/US2019/041562, filed on Jul. 12, 2019.
(60) Provisional application No. 62/697,179, filed on Jul. 12, 2018.

(51) Int. Cl. G01N 33/68; C12N 15/10; G16B 15/00

(52) U.S. Cl. CPC G01N 33/6818; C12N 15/1065; G16B 15/00; G01N 2458/00; G01N 2570/00

(57) ABSTRACT

The present disclosure provides methods for molecular neighborhood detection of molecules, such as by iterative proximity ligation or split-and-pool methods for obtaining positional information.

This application is a continuation of U.S. patent application Ser. No. 17/146,165, filed Jan. 11, 2021, as a continuation of International Application No. PCT/US2019/041562, filed Jul. 12, 2019, which claims the benefit of U.S. Provisional Patent Application No. 62/697,179, filed Jul. 12, 2018, the entire contents of each of which is incorporated herein by reference in their entirety.

### INCORPORATION OF SEQUENCE LISTING

This application contains a Sequence Listing XML, which has been submitted electronically and is hereby incorporated by reference in its entirety. Said XML Sequence Listing, created on Dec. 2, 2024, is named UTSBP1195USC1.xml and is 97,690 bytes in size.

### BACKGROUND

This invention was made with government support under Grant no. OD009572 and R35 GM122480 awarded by the National Institutes of Health and Grant no. N66001-14-2-4051 awarded by the Space and Naval Warfare Systems Center, Pacific. The government has certain rights in the invention.

### 1. Field

The present disclosure relates generally to the fields of chemistry and molecular biology. More particularly, it concerns methods for identifying which molecules share the same neighborhood in space, and the resulting approaches that allow for mapping these molecules positions in space or, in other variations, detecting and identifying molecules using oligonucleotides.

### 2. Description of Related Art

Aside from carrying genetic information in organisms, DNA has proven utility across a broad field of applications. As the mastery of chemistries and enzymes to manipulate DNA has deepened, so has the breadth of its utility. One of the most powerful developments contributing to this trend has been the rapid growth in high-throughput DNA sequencing (Shendure et al., 2017), now providing a remarkable stream of new information about the world.

DNA barcoding—the incorporation of specific, pre-designed DNA sequences into molecules as an approach to identify them or copies of them across experiments—has been a key innovation for accelerating the throughput of sequencing (Buschmann and Bystrykh, 2013) and as such sits at an interesting intersection of biology and information theory. The most basic application of DNA barcoding is to label and thus track individual molecules as they are manipulated. This concept has been expanded to obtain information beyond tracking particle identities, for example to identifying their interaction partners (Soderberg et al., 2017) and spatial positions (Schaus et al., 2017). However, there is an unmet need for improved methods of detecting single molecules, such as proteins or peptides, by using the spatial positions of the molecules.

### SUMMARY

In certain embodiments, the present disclosure provides methods of interrogating molecular neighborhoods. A sample of dilute proteins can be interrogated for positional information, such as by two-round techniques including iterative proximity ligation (IPL) or split-and-pool labeling, using oligonucleotide probes. The IPL method can be used to define a neighborhood by wherever an oligonucleotide probe can reach while the split-and-pool method can be used to define the neighborhood by a set of oligonucleotides linked to each other through a physical intermediate.

In one embodiment, there is provided a method for performing iterative proximity ligation (IPL) comprising attaching oligonucleotide tags (e.g., deoxyribonucleic acid (DNA) tags) to a plurality of molecules in a sample, wherein the oligonucleotide tags comprise (1) a functional group for attachment to a molecule, (2) a primer site, (3) a unique barcode, and (4) a 5′ cleavage half-site or a 3′ cleavage half-site; ligating oligonucleotide tags with a 5′ cleavage half-site to oligonucleotide tags with a 3′ cleavage half-site which are held in proximity, thereby generating one or more barcode pairs; extending the primer in one of the oligonucleotide tags of the one or more barcode pairs to generate duplicates of the barcode pairs; and adding a catalyst for cleavage to separate the oligonucleotide tags.

In some aspects, the oligonucleotide tags are single-stranded or double-stranded. In certain aspects, the functional group is selected from the group consisting of succinimidyl ester, iodoacetamide, maleimide, amines, 4-phenyl-3H-1,2,4-triazole-3,5 (4H)-dione (PTAD), 2,4-dinitrobenzenesulfenyl chloride, and a thiol.

In some aspects, ligating is reversible. In particular aspects, the tags are not conjugated to a fluorescent label. In certain aspects, ligating comprises adding a protein ligase and a bridging oligonucleotide. In some aspects, the protein ligase is a blunt-end ligase. In certain aspects, the protein ligase is T4 DNA ligase, T4 RNA ligase, Taq DNA ligase, T3 DNA ligase, or T7 DNA ligase. In some aspects, ligating comprises chemical ligation. In particular aspects, chemical ligation comprises using click chemistry, carbodiimides, or phosphorothioate-iodide coupling.

In certain aspects, the bridging oligonucleotide comprises a first region of complementarity to the tag with the 5′ cleavage half-site and a second region of complementarity to the tag with the 3′ cleavage half-site. In some aspects, the catalyst for cleavage is a sequence-specific endonuclease. In certain aspects, the sequence-specific endonuclease is a restriction enzyme, zinc finger nuclease, sequence-specific ribozyme, or CRISPR-associated endonuclease. In some aspects, the catalyst for cleavage is an oligonucleotide modified with a chemical cleavage reagent. In certain aspects, the chemical cleavage agent is iron: EDTA. In some aspects, the oligonucleotide tags with the 5′ cleavage half-site and oligonucleotide tags with the 3′ cleavage half-site are added to the sample at a 1:1 ratio before attaching the oligonucleotide tags.

In some aspects, a round of IPL generates multiple barcode pairs. In particular aspects, each round of IPL generates at least 2×10¹⁰ (e.g., at least 2×10¹¹, 2×10¹², 2×10¹³, or 2×10¹⁴) duplicate barcode pairs.

In additional aspects, the method further comprises performing at least one additional round of IPL on said sample to obtain multiple duplicate barcode pairs. In particular aspects, 2, 3, 4, 5, or more rounds of IPL are performed. In particular aspects, the one or more rounds of IPL are performed in a single reaction tube.

In some aspects, the molecule is a protein, protein complex, peptide, antibody, carbohydrate, nucleic acid, cell or a receptor. In certain aspects, IPL is performed on a molecular surface or in solution. In some aspects, the molecular surface is organic or inorganic. In specific aspects, the molecular surface comprises crystals, plastic, or metal. In particular aspects, the cell is a mammalian cell, cancer cell, or cell infected with an infectious agent. In certain aspects, the infectious agent is HIV.

In additional aspects, the method further comprises amplifying the duplicate barcode pairs. In some aspects, amplifying comprises performing polymerase chain reaction (PCR). In some aspects, the method further comprises performing sequencing on the duplicate barcode pairs. In certain aspects, the method further comprises identifying and quantifying one or more molecules in the sample. In certain aspects, identifying and quantifying comprise generating a graph from the sequence of the duplicated barcode pairs. In some aspects, generating a graph comprises interpreting each barcode as a node and each barcode pair as an edge. In some aspects, the graph positions the molecules in the sample at a at nanometer or micrometer scale. In particular aspects, the method further comprises simulating Erdos-Renyi random graph connectivity, random geometric graph connectivity, or spring-layout algorithms to recover spatial information for one or more molecules in the sample. In certain aspects, the method further comprises applying Graphviz Neato implementation of a Kamada-Kawai algorithm to the spring-layout algorithm. In some aspects, the Kamada-Kawai algorithm is a modified variant that performs local-only spring energy minimization. In certain aspects, the Kamada-Kawai algorithm is applied followed by a second round using the modified Kamada-Kawai algorithm.

In another embodiment, there is provided a composition comprising a oligonucleotide tag comprising (1) a functional group for attachment to the molecule, (2) a primer site, (3) a unique barcode, and (4) a 5′ restriction half-site ligated to a oligonucleotide tag comprising (1) a functional group for attachment to the molecule, (2) a primer site, (3) a unique barcode, and (4) a 3′ restriction half-site. In some aspects, each of the oligonucleotides tags are attached to a molecule.

In yet another embodiment, there is provided a method of determining the spatial position of an individual molecule(s) comprising obtaining a sample comprising a plurality of individual molecules; applying multiple rounds of IPL according to claim 1 on said sample to generate a plurality of duplicate barcode pairs; performing next-generation sequencing on the plurality of duplicate barcode pairs; and applying pairwise proximity to determine the spatial position of the individual molecule(s).

In some aspects, the sample is a biological sample. In certain aspects, the biological sample is saliva, urine, blood, or whole tissue. In certain aspects, the method does not comprise using microscopy. In certain aspects, the individual molecule is not bound to a solid support.

In some aspects, the molecule is a protein, protein complex, peptide, antibody, carbohydrate, nucleic acid, cell, signaling domain, or a receptor. In certain aspects, the antibody is bound to another antibody or a ligand. In particular aspects, the molecule is a protein or peptide. In specific aspects, the protein or peptide is a biomarker for cancer or an infectious disease. In some aspects, the protein or peptide is in solution. In some aspects, amino acids in the protein or peptide are labeled with amino-acid specific oligonucleotide tags each comprising a unique barcode. In particular aspects, the amino acids are lysine, cysteine, glutamic acid, aspartic acid, tyrosine, tryptophan, histidine, or any combination thereof. In some aspects, the amino acids comprise post-translationally modified side chains. In specific aspects, the post-translationally modified side chains comprise phosphorylation, glycosylation, methylation, citrullination, or any combination thereof.

In additional aspects, the method further comprises determining the identity and quantity of the protein or peptide. In some aspects, determining the identity of the protein or peptide comprises obtaining an amino acid pattern from the next-generation sequencing and comparing the amino acid pattern to the proteome of an organism to identify the protein or peptide.

In particular aspects, the second step is performed in a single reaction tube. In some aspects, the sample comprises from 2 to 10¹² proteins, such as at least 3, 4, 5, 10, 15, 20, 50, 100, 500, 10³, 10⁴, 10⁵, 10⁶, 10⁷, 10⁸, 10⁹, 10¹⁰, 10¹¹, 10¹² or more proteins. In some aspects, the sample is diluted before DNA sequencing.

In additional aspects, the method further comprises digesting the protein with an enzyme. In certain aspects, the enzyme site-specifically digests the protein. In particular aspects, the enzyme is trypsin. In some aspects, the digestion does not remove the N-terminal amino acid.

In some aspects, the method further comprises applying a second set of IPL rounds to the digested protein. In certain aspects, the method further comprises reconciling the duplicate barcode pairs from each set of IPL rounds to identify the protein.

In some aspects, the molecules are nanobeads. In certain aspects, the method further comprises determining the shape and size of the nanobeads.

A further embodiment provides a method of determining the identity of a protein or peptide comprising obtaining a sample comprising a plurality of proteins peptides, or a combination thereof; applying multiple rounds of IPL according to claim 1 on said sample to generate a plurality of duplicate barcode pairs; performing next-generation nucleic acid sequencing on the plurality of duplicate barcode pairs; and obtaining an amino acid pattern for a single protein or peptide from the next-generation nucleic acid sequencing and comparing the amino acid pattern to the proteome of an organism to identify the protein or peptide.

In some aspects, the sample is a biological sample. In certain aspects, the biological sample is saliva, urine, blood, or whole tissue. In certain aspects, the protein or peptide is a biomarker for cancer or an infectious disease.

In certain aspects, the method does not comprise using microscopy. In some aspects, the individual molecule is not bound to a solid support. In certain aspects, the protein or peptide is in solution. In some aspects, amino acids in the protein or peptide are labeled with amino-acid specific oligonucleotide tags each comprising a unique barcode. In particular aspects, the amino acids are lysine, cysteine, glutamic acid, aspartic acid, tyrosine, tryptophan, histidine, or a combination thereof. In specific aspects, the amino acids comprise post-translationally modified side chains. In some aspects, the post-translationally modified side chains comprise phosphorylation, glycosylation, methylation, citrullination, or a combination thereof.

In some aspects, the second step is performed in a single reaction tube. In some aspects, the sample comprises from about 2 to about 10¹² proteins, such as at least 3, 4, 5, 10, 15, 20, 50, 100, 500, 10³, 10⁴, 10⁵, 10⁶, 10⁷, 10⁸, 10⁹, 10¹⁰, 10¹¹, or 10¹² proteins. The sample is diluted before sequencing.

In additional aspects, the method further comprises digesting the protein with an enzyme. In some aspects, the enzyme site-specifically digests the protein. In certain aspects, the enzyme is trypsin. In some aspects, the digestion does not remove the N-terminal amino acid.

In some aspects, the method further comprises applying a second set of IPL rounds to the digested protein. In certain aspects, the method further comprises reconciling the duplicate barcode pairs from each set of IPL rounds to identify the protein.

In another embodiment, there is provided a method for uniquely labeling a protein comprising obtaining a sample comprising a plurality of proteins; attaching oligonucleotide tags comprising unique amino acid-specific barcodes to the plurality of proteins; splitting the sample into three or more compartments; labeling the plurality of proteins with a compartment-specific oligonucleotide tag; pooling the sample from the three or more compartments and re-splitting the sample into three or more compartments; and attaching a second compartment-specific oligonucleotide tag to the plurality of proteins, thereby obtaining a uniquely labeled protein.

In additional aspects, the method further comprises one or more rounds of pooling the sample, splitting the sample, and attaching additional compartment-specific oligonucleotide tags to obtain uniquely labeled proteins. In some aspects, the amino acid-specific barcode is specific for lysine, cysteine, glutamic acid, aspartic acid, tyrosine, tryptophan, or histidine. In certain aspects, the amino acids in the plurality of proteins comprise post-translationally modified side chains. In some aspects, the post-translationally modified side chains comprise phosphorylation, glycosylation, methylation, citrullination, or a combination thereof. In certain aspects, the amino acid-specific barcodes comprise a functional group to attach the barcode to the amino acid. In some aspects, the functional group is selected from the group consisting of succinimidyl ester, iodoacetamide, maleimide, amines, 4-phenyl-3H-1,2,4-triazole-3,5(4H)-dione (PTAD), 2,4-dinitrobenzenesulfenyl chloride, and a thiol.

In some aspects, the method further comprises digesting the protein with an enzyme. In certain aspects, the enzyme site-specifically digests the protein. In particular aspects, the enzyme is trypsin. In some aspects, the digestion does not remove the N-terminal amino acid.

In some aspects, the method further comprises applying a second set of IPL rounds to the digested protein.

In yet another embodiment, there is provided a method of determining the identity of a protein comprising obtaining a sample comprises a plurality of uniquely labeled proteins according to the present embodiments and aspects thereof; performing next-generation sequencing on the plurality of duplicate barcode pairs; and obtaining an amino acid pattern for a single protein or peptide from the next-generation sequencing and comparing the amino acid pattern to the proteome of an organism to identify the protein or peptide.

In certain aspects, the sample is a biological sample. In particular aspects, the biological sample is saliva, urine, blood, or whole tissue.

In particular aspects, the method does not comprise using microscopy. In some aspects, the individual molecule is not bound to a solid support. In certain aspects, the protein or peptide is a biomarker for cancer or an infectious disease. In specific aspects, the protein or peptide is free in solution. In some aspects, amino acids in the protein or peptide are labeled with amino-acid specific oligonucleotide tags each comprising a unique barcode. In some aspects, the sample comprises from about 2 to about 10¹² proteins, such as at least 3, 4, 5, 10, 15, 20, 50, 100, 500, 10³, 10⁴, 10⁵, 10⁶, 10⁷, 10⁸, 10⁹, 10¹⁰, 10¹¹, or 10¹² proteins. In certain aspects, the sample is diluted before sequencing.

In additional aspects, the method further comprises digesting the protein with an enzyme. In some aspects, the enzyme site-specifically digests the protein. In some aspects, the enzyme is trypsin. In particular aspects, the digestion does not remove the N-terminal amino acid.

A further embodiment provides a method of detecting a molecular neighborhood comprising obtaining a dilute solution of proteins; generating positional information for amino acids using a two-round technique; analyzing the positional information to obtain the molecular neighborhood. In certain aspects, the method further comprises identifying a protein or protein complex which comprises amino acids in close proximity to each other. In some aspects, the method further comprises identifying at least a second protein or protein complex. In certain aspects, the two-round technique is IPL or split-and-pool labeling. In particular aspects, the two-round technique is a method according to the present embodiments and aspects thereof. In some aspects, the two-round technique comprises proteolysis between the first and second round.

As used herein, “essentially free,” in terms of a specified component, is used herein to mean that none of the specified component has been purposefully formulated into a composition or is present as a contaminant or in trace amounts. The total amount of the specified component resulting from any unintended contamination of a composition is therefore well below 0.05%, preferably below 0.01%. In another embodiment, a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. The detailed description and the specific examples, while indicating preferred embodiments of the disclosure, are given by way of illustration, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1D: Iterative Proximity Ligation Scheme Overview. A, top: Each iterative proximity ligation (IPL) oligonucleotide is a single-stranded deoxyribonucleic acid (DNA) synthesized to contain four components: a functional group for attachment, a primer site, a unique barcode sequence, and a restriction half-site. A, bottom: The oligonucleotide population is an approx. 1:1 mixture of two opposite polarities (A, bottom) such that they can be ligated using e.g. T4 ligase in the presence of a short bridging oligo. Primer and restriction site sequences are shared across each polarity; barcodes are unique across the entire population. (B) One round of IPL comprises ligating two oligonucleotides of opposite polarity, extending one of the primers to duplicate the barcode pair into the solution, and restriction to revert both oligonucleotides to their un-ligated state. (C) Presence of multiple neighboring oligonucleotides allows a different barcode pair to be recorded each round. IPL acts on a bulk population of oligonucleotides in parallel, with each round recording many barcode pairs at once. (D) After multiple IPL rounds, all recorded barcode pairs are sequenced by next-generation DNA sequencing. Interpretation of sequenced pairs as a graph-with each individual barcode treated as a node and each pair treated as an edge-leads to a rich application space.

FIGS. 2A-2D: The number of possible nodes and possible edges recovered increases with the number of IPL rounds and ligation efficiency during each round. For each combination of IPL rounds and ligation efficiency parameters, 100 independent replicates were simulated of patterning a random “W” shape via a Poisson process, followed by iterative simulation of ligations between deposited oligonucleotides. The number of nodes and edges were then tallied participating in the largest IPL graph component as a percent of all possible participating nodes and edges under an ideal scenario (i.e. where all nodes would ligate with certainty if they were within reach of two complimentary oligonucleotides). (A) Percent of nodes participating in the largest component, averaged across 100 simulations. (B) Relative standard deviation across the 100 simulations in A. (C) Percent of possible edges participating in the largest component, averaged across the 100 simulations. (D) Relative standard deviation across the 100 simulations in C. Square indicates parameters used for simulation, with an average of 98% of nodes and 44% of possible edges participating in the largest component.

FIG. 3: Six IPL rounds with a 0.3 ligation efficiency still recovers the original pattern overall. Note that nodes not connected to the largest graph component are not shown, as their position cannot be ascertained relative to the bulk of their connected peers.

FIGS. 4A-4B: IPL single molecule proteomics labeling scheme. (A) Cysteines, lysines, tryptophans, and carboxylates can be selectively labeled with barcoded oligonucleotides via handles specific to each amino-acid's side chain. The oligonucleotides encode the identity of their target amino-acid species and a random sequence unique to each individual amino acid amongst a large mixture of proteins. (Note: primers and half-restriction sites omitted from diagram for clarity.) (B) This chemistry is applied in bulk solution to a protein mixture, denaturing each protein and barcoding its amino acids.

FIGS. 5A-5C: Partial amino acid composition is sufficient to uniquely identify proteins. A: Performing IPL on a dilute population of labeled proteins yields a tally of labeled amino acids for each protein. This partial compositional information is sufficient to identify a substantial portion of the E. coli and H. sapiens proteomes using three-label combinations. “[DIE]” indicates Asp/Glu labels. Number proteins identified is above each bar. The E. coli and H. sapiens proteomes used for the simulation contain 4297 and 19988 proteins respectively. B: After applying an initial sequence of IPL rounds on whole proteins, they can be digested by trypsin and another sequence of IPL rounds can be repeated on the tryptic peptides. Oligonucleotide pairs from the first and second sets of IPL rounds can be reconciled because all barcodes are unique across the entire protein population. The additional compositional information about tryptic subsequences in the second stage significantly boosts identification. C: Substantial proportions of the proteome can be identified even at reduced label efficiencies. Labeling efficiencies for Cys and Asp/Glu have experimentally been shown to be quantitative.

FIG. 6: Testing reversible ligation on solid support. Left and right probes (Table 1) were mixed in approx. a 1:1 ratio and then incubated with magnetic streptavidin beads. After the first ligation, beads were mixed well and randomly sampled by pipette for qPCR. The remaining beads were washed and incubated with a restriction enzyme, and sampled again. A second ligation was carried out and sampled. All ligations and restrictions required addition of a connector complementary to the restriction/ligation site (Table 1).

FIGS. 7A-7C: Quantitation of ligated probes following ligation & restriction. A: Beads with attached probes were sampled after the first ligation, restriction, and second ligation for each of six replicates. The number of ligated probes were quantified in each after each reaction using qPCR. B: Each replicate's C_{q} increased significantly after restriction, implying a significant decrease in remaining ligated pairs. C: All qPCR reactions were run on a PAGE gel with a size marker to confirm the 65 bp product size expected from the oligonucleotide sequences (Table 1).

FIG. 8: Schematic depicting split-sequencing single molecule proteomics method for barcoding and identifying amino acids within a protein.

FIG. 9: The Kamada-Kawai algorithm regards graph distance between two nodes as the desirable Euclidean distance between them. Concave geometries lead to nodes being much closer in Euclidean space (solid line with arrows) than in graph space (dashed line). This discrepancy forces nodes apart during graph layout. This problem was corrected for by taking the recovered layout and re-feeding it into a modified variant of the KK algorithm that performs local-only spring energy minimization instead of a global minimization. This significantly decreased large-scale distortions.

FIGS. 10A-10B: DNA as might be deposited on a surface with various photolithigraphed shapes. (FIG. 10B) Photolithigraphed shapes of analogue recovered under imperfect conditions.

FIGS. 11A-11B: (A) Double label azidolysine peptide design. (SEQ ID NO: 49) (B) Double label azidolysine peptide with conjugated oligos.

FIGS. 12A-D: (A) Outline of control (non-split-and-pool) labeling & ligation experiments. (Step 1) Label double-AzK peptide with hexynyl oligo via CuAAC. Isolate doubly-labeled peptide by PAGE. (Step 2) Perform three rounds of barcode ligation onto labeled peptide and isolate the product. (Step 3) PCR amplify ligated barcodes and confirm sequence. (B) Control (non-split-and-pool) oligonucleotide designs. (SEQ ID NOS: 6-18) (C) Doubly labeled peptide isolated by PAGE. (D) Confirming presence of peptide by streptavidin gel shift.

FIGS. 13A-B: (A) Each round of ligation by PAGE gel analysis. (B) PCR of double labeled peptide.

FIGS. 14A-14E: (A) Barcodes for split-and-pool method. “N”s represent UMIs. (SEQ ID NOS: 19-48) (B) Split-and-pool ligated product isolated by PAGE. (C-D) Labeling cysteines using a thiol/maleimide reaction. (SEQ ID NOS: 50-51) (E) Ligation and PCR on thiol/maleimide labeled products.

FIGS. 15A-15C: (A) Serially diluted qPCR of purified product. (B) Amplification and Sanger sequencing of purified product. (SEQ ID NOS: 52-55) (C) Sequence quality curves.

FIGS. 16A-16D: (A) Distribution of the alignment scores. (B) Alignment scores for combination. (C) Representative alignments with scores ≥260 (i.e. perfect alignment score for the product). (D) Representative alignments with scores between 240 and 250 (i.e. suboptimal alignments).

### DETAILED DESCRIPTION

In certain embodiments, the present disclosure provides a novel approach for using nucleic acid sequencing to report on the relative spatial distributions of nucleic acid molecules (e.g., deoxyribonucleic acid (DNA) molecules), as measured by combining molecule-specific nucleic acid barcodes with a proximity ligation assay designed to be performed multiple, consecutive times. Such nucleic acid barcodes may be DNA or ribonucleic acid (RNA) barcodes. Such nucleic acid barcodes may be oligonucleotides having lengths of at least about 2, 3, 4, 5, 6, 7, 8, 9, 10, 20, 30, 40, 50, or more nucleic acid bases. Importantly, the present methods do not require the use of microscopy to detect or identify a molecule. By not relying on visual information, the present methods can sidestep limitations of traditional microscopy to obtain information about individual molecules in parallel, such as millions of molecules in parallel. In addition, the present methods do not depend on particle tracking, occlusion, or the complexity of the mixture.

The present studies demonstrated using computer simulations how the spatial positions of many DNA molecules can be simultaneously recovered using DNA sequence information. The methods provided herein may be used for determining object shapes (e.g., at a micrometer (or micron) scale) and single-molecule proteomics. In particular, the present studies experimentally demonstrate proof-of-principle iterative ligation on a simple model system.

The present methods employ DNA (or other nucleic acid, such as RNA) proximity ligation from a single readout per oligonucleotide pair to multiple reversible, iterative ligations re-using the same oligonucleotide molecules. Thus, certain embodiments of the present disclosure provide methods of using iterative proximity ligation (IPL) to capture multiple ligation events for each oligonucleotide and its various neighbors. Thus, the present methods can provide more detailed knowledge about the relative positions of the molecules as compared to single, irreversible ligation events. By integrating a unique DNA barcode into each participating oligonucleotide, a read-out of these ligation events may be obtained and thus provides the positional information contained therein in a high throughput manner using next-generation DNA sequencing.

Graph theory may be applied to the IPL sequencing results. This can be applied to multiple applications as described herein. In one method, the present studies show that geometric patterns of objects labeled by DNA can be obtained and single-molecule proteomics can be performed. In particular, it was demonstrated that letter patterns photolithographed onto slide-surfaces can be recovered using IPL sequencing data, illustrating how the present technique maps complex spatial configurations into DNA sequences and then-using this sequence information-recovers them. In another application, it was shown that IPL can identify and quantitate a large proportion of the E. coli proteome at single-molecule resolution even under suboptimal chemical and enzymatic reactivities.

Accordingly, methods are provided herein for assigning identity to individual molecules or physical positions using barcoded DNA molecules. Recording their pairwise proximities by iterative proximity ligation can reveal properties of the labeled objects. Specifically, iterative proximity ligation establishes a direct relationship between spatial position and sequence information. In one example of IPL for proximity detection, IPL comprising repetitive DNA ligation and digestion may be applied on a population of molecules. The population of IPL oligonucleotides can be ligated, restricted, and ligated again, with qPCR quantitating the efficiency of each step.

In one application of IPL, graph theory may be used to recover oligonucleotide positions, such as by using spring layout algorithms. The present methods have low error rates in positional recovery. For example, neighborhoods (e.g., about 1 μm diameter) can be recovered with an average oligonucleotide positional error less than the length of a ligated oligonucleotide pair. On scales substantially larger than individual oligonucleotides, three-dimensional shapes can be recovered using DNA sequencing.

Another application of IPL provided herein comprises single-molecule proteomics. This may be used to identify a peptide(s) or a protein(s) from a cell. A peptide may be a polypeptide. In this case, spatial proximity is informative because proximity of oligonucleotide-labeled amino-acids implies they share a polypeptide chain, allowing IPL to capture protein compositional information. The present studies showed that such compositional information is sufficient to uniquely identify a large proportion of the E. coli proteome even under imperfect labeling chemistries. Further methods provided herein concern detecting protein complexes by labeling individual proteins via antibodies or other tagging mechanisms. Thus, protein complex composition can be recovered at single-molecule resolution.

### I. Molecular Neighborhood Detection

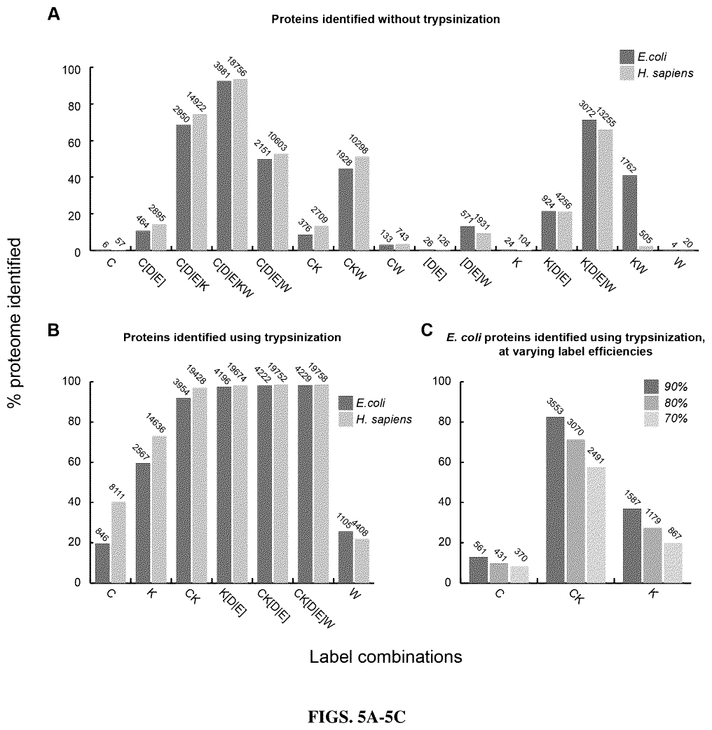

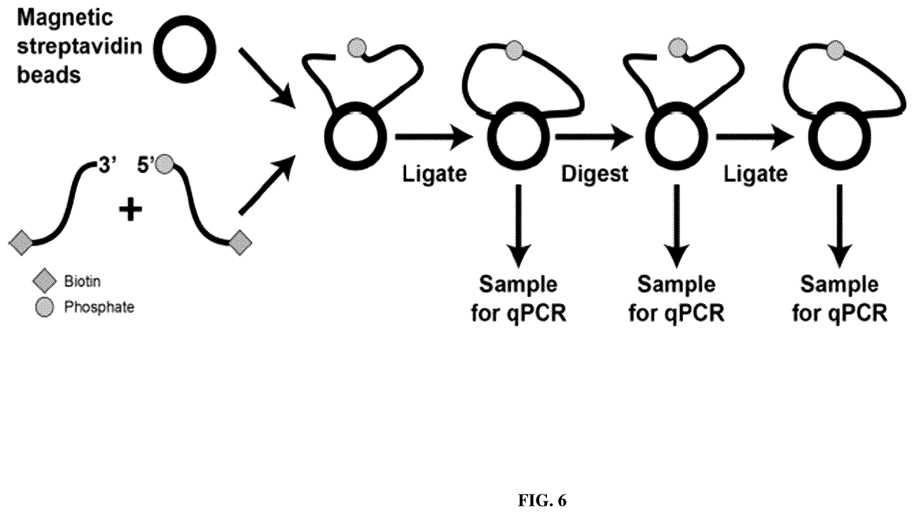

In sufficiently dilute solutions of proteins, proteins are far enough apart that each one's amino-acids are unlikely to be near the amino-acids belonging to other protein molecules. Therefore, a molecular neighborhood detection technique can infer which amino acids belong to the same protein by virtue of their proximity to each other—as they comprise the same molecule, i.e. the same neighborhood—while at the same time being far away from amino acids of other molecules. By detecting the amino acids in each neighborhood (each molecule), the number of each amino acid (e.g. Lys, Cys, etc.) belonging to that molecule can be counted. As shown in FIG. 5, this compositional information is sufficient to uniquely identify a large proportion of proteins in, for example, human and E. coli proteomes. Thus, in certain embodiments, there are provided methods for molecular neighborhood detection. The method can comprise obtaining a dilute solution of molecules, such as peptides, proteins, or protein complexes, obtaining positional information for amino acids within close proximity to each other and identifying the molecule which comprises said amino acids. Thus there are provided herein methods for detecting the molecular neighborhoods of amino acids which can be used to identify proteins or other molecules at single-molecule resolution. The present methods can be extended to other molecules where compositional information is present. For example, the same methods can be applied to protein complexes, labeling and counting individual proteins, and thus obtaining the stoichiometry of that complex.

One approach provided herein for detecting molecular neighborhoods, specifically to obtain compositional information, is IPL which can define a neighborhood by wherever an oligo probe can read. In another method provided herein, referred to as split-and-pool, can be used to define a neighborhood as a set of oligos linked to each other through a physical intermediate. The two-round compositional information, whether obtained by IPL or split-and-pool, can be used for neighborhood detection.

The compositional information obtained from the IPL or split-and-pool methods can then be analyzed using bioinformatics to identify one or more molecules, such as peptides, proteins, or protein complexes, in a sample, such as for proteomics analysis.

“Subject” and “patient” refer to either a human or non-human, such as primates, mammals, and vertebrates. In particular embodiments, the subject is a human.

“Sample” generally refers to a material obtained or isolated from a fresh or preserved biological sample or synthetically-created source that contains molecules of interest. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals.

Samples can include, for example, a bodily fluid from a subject, including amniotic fluid surrounding a fetus, aqueous humor, bile, blood and blood plasma, cerumen (earwax), Cowper's fluid or pre-ejaculatory fluid, chyle, chyme, female ejaculate, interstitial fluid, lymph, menses, breast milk, mucus (including snot and phlegm), pleural fluid, pus, saliva, sebum (skin oil), semen, serum, sweat, tears, urine, vaginal lubrication, vomit, feces, internal body fluids including cerebrospinal fluid surrounding the brain and the spinal cord, synovial fluid surrounding bone joints, intracellular fluid (the fluid inside cells), and vitreous humour (the fluids in the eyeball). In particular aspects, the sample is a blood sample, such as a peripheral whole blood sample, or a fraction thereof. The sample may be whole, unfractionated blood. The blood sample can be at least about 0.02, 0.03, 0.04, 0.05, 0.06, 0.07, 0.08 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0 milliliters (mL), or more. The sample can be obtained by a health care provider, for example, a physician, physician assistant, nurse, veterinarian, dermatologist, rheumatologist, dentist, paramedic, or surgeon. The sample can be obtained by a research technician. More than one sample from a subject can be obtained.

### A. Compositional Information

The compositional information of the present methods can be obtained by using IPL's repetitive DNA ligation and digestion. The method can comprise repeated ligation and restriction followed by DNA sequencing of the oligonucleotides. DNA oligonucleotides may be attached to single molecules and two oligonucleotides in sufficient proximity may then be ligated to each other. The pair of oligonucleotides may then be duplicated in solution and cleaved to un-ligate or separate the two oligonucleotides. For the next round, other neighboring oligonucleotides can pair and be duplicated and un-ligated. Multiple round of this IPL method can be performed to obtain spatial information on the individual molecules.

In some aspects, the compositional information may be obtained by the split-and-pool method in which the molecules may be labeled with tags in a split-and-pool method. For example, the molecules may be proteins or peptides labeled with both an amino acid identifier and a unique identifier in a sample. In this method, the sample is split into compartments, such as a number of wells, and ligated to an additional well-specific identifier. The compartments may be a well, microwell, or microfluidic droplet. Then, the sample is pooled and re-split before the addition of an additional, well-specific barcode to all of the conjugated oligonucleotides. The samples can then be pooled, and re-allotted to different wells, and a second, well-specific barcode is appended. Additional rounds of pooling, splitting, and ligation may be carried out in order to further diversify the combinatorial, ligated barcodes to the point where there is a unique combinatorial, ligated barcode for each protein or peptide. The key result is that each protein has its own unique barcode, and a copy of this barcode has been appended to each of the protein's labeled residues. Reading out the well codes (underlined) distinguishes the two proteins, and thus also indicates which residues go together on these proteins. The individual residues on each protein can be distinguished by their “unique barcode” sequences, and hence the number of each kind of amino acid (i.e., cysteine or lysine) that protein contains can be counted.

The terms “barcode,” “unique tag” or “unique identifier” are used interchangeably herein to refer to a unique nucleotide sequence that is used to identify a single molecule or population of molecules. Barcodes can be linked to a target molecule of interest and used to trace back the amplicon. The barcode may be an oligonucleotide of 5-40 nucleotides, particularly 8-12 nucleotides, such as 8, 9, 10, 11, 12, 13, 14, or 15 nucleotides in length. In particular aspects, the barcode is comprised of random (e.g., degenerate) nucleotides.

The barcode or unique tag may be assigned to an individual molecule and then manipulated to determine if the tags are within proximity to each other. The DNA oligonucleotide may comprise a functional group, a primer site, a unique barcode, and a cleavage half-site, such as a restriction enzyme half-site. The cleavage half-site may be at the 5′ or 3′ end of the oligonucleotide. The cleavage site may be for an endonuclease, such as a blunt-end enzyme including but not limited to AanI, Acc16I, AccBSI, AccII, AcvI, AfaI, AfeI, AjiI, AleI, AluBI, Alul, Aor51HI, Asp700I, Ball, BbrPI, BmcAI, BmgBI, BmiI, BoxI, BsaAI, BsaBI, Bse8I, BseJI, Bsh1236I, BshFI, BsnI, Bsp68I, BspANI, BspFNI, BspLI, BsrBI, BssNAI, Bst1107I, BstBAI, BstC8I, BstFNI, BstPAI, BstSNI, BstUI, BstZ17I-HF, BsuRI, BtrI, BtuMI Cac8I, CviJI, CviKI-1, DinI, DpnI, Dral, Ecl136II, Eco105I, Eco147I, Eco32I, Eco47III, Eco53kI, Eco72I, EcoICRI, EcoRV, EcoRV-HF, Egel, Ehel, Fail, FspAI, FspI, GlaI, HaelII, HincII, HindII, HpaI, Hpyl66II, Hpy8I, HpyCH4V, KspAI, MalI, MbiI, MlsI, MluNI, MlyI, Mox20I, MroXI, MscI, MsII, Msp20I, MspA1I, MssI, MvnI, NaeI, NlaIV, NruI, NruI-HF, NsbI, OliI, PceI, PdiI, PdmI, PmaCI, PmeI, PmlI, Ppu21I, PshAI, PsiI, PspCI, PspN4I, PvuII, PvuII-HF, RruI, RsaI, RseI, ScaI, ScaI-HF, SchI, SfoI, SmaI, SmiI SmiMI, SnaBI, SrfI, SseBI, SspI, SspI-HF, StuI, SwaI, XmnI, ZraI, and ZrmI

The functional group may be succinimidyl ester, iodoacetamide, maleimide, amines, 4-phenyl-3H-1,2,4-triazole-3,5(4H)-dione (PTAD), 2,4-dinitrobenzenesulfenyl chloride, a thiol, or any combination thereof. The oligonucleotides with cleavage sites of opposite polarity (i.e., 5′ and 3) may be ligated by a protein ligase, such as T4 DNA ligase, T4 RNA ligase, Taq DNA ligase, T3 DNA ligase, or T7 DNA ligase.

The present methods may be used to determine the spatial positions and identities of individual molecules, such as single molecules within a population of molecules. The molecules may comprise a protein, protein complex, peptide, antibody, carbohydrate, nucleic acid, cell, signaling domain, lipid, carbohydrate, or a receptor. The individual molecules may be identified on a large-scale, such as from a sample with a population of molecules.

Proteins or peptides may be labeled at amino acid residues, such as cysteine, aspartic acid, or glutamic acid. Other residues may be labeled including, but not limited to, lysine, cysteine, glutamic acid, aspartic acid, tyrosine, tryptophan, histidine, or any combination thereof. The amino acids may be modified with phosphorylation, glycosylation, methylation, citrullination, or any combination thereof.

### A. Sequencing

The term “primer” or “oligonucleotide primer” as used herein, refers to an

oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification, but may alternatively be double-stranded. If double-stranded, the primer can first be treated to separate its strands before being used to prepare extension products. This denaturation is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

“Polymerase chain reaction,” or “PCR,” generally refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following processes: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each process in a thermal cycler instrument. Particular temperatures, durations at each process, and rates of change between processes depend on many factors, e.g., exemplified by the references: McPherson et al., editors, PCR: A Practical Approach and PCR2: A Practical Approach (IRL Press, Oxford, 1991 and 1995, respectively).

Any technique for sequencing nucleic acids can be used in the methods of the present disclosure. DNA sequencing techniques include dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during polymerization, and SOLID sequencing. The input RNA may be 10%, 15%, 30%, or higher.

In certain embodiments, the sequencing technique used in the methods of the provided disclosure generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, at least 1,000,000 reads per run, at least 2,000,000 reads per run, at least 3,000,000 reads per run, at least 4,000,000 reads per run at least 5000,000 reads per runs at least 6,000,000 reads per run at least 7,000,000 reads per run at least 8,000,000 reads per runs at least 9,000,000 reads per run, at least 10,000,000 reads per run, or more.

In certain embodiments, the sequencing technique used in the methods of the provided disclosure can generate at least about 30 bp, about 40 bp, about 50 bp, about 60 bp, about 70 bp, about 80 bp, about 90 bp, about 100 bp, about 110, about 120 by per read, about 150 bp, about 200 bp, about 250 bp, about 300 bp, about 350 bp, about 400 bp, about 450 bp, about 500 bp, about 550 bp, about 600 bp, about 700 bp, about 800 bp, about 900 bp, about 1,000 bp, or more per read. For example, the sequencing technique used in the methods of the provided disclosure can generate at least about 30 bp, 40 bp, 50 bp, 60 bp, 70 bp, 80 bp, 90 bp, 100 bp, 110 bp, 120 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1,000 bp, or more by per read.

### 1. HiSeq™ and MiSeq™ Sequencing

In particular aspects, the sequencing technologies used in the methods of the present disclosure include the HiSEQ™ system (e.g., HiSEQ2000™ and HiSEQIOOO™) and the MiSEQ™ system from Illumina, Inc. The HiSEQ™ system is based on massively parallel sequencing of millions of fragments using attachment of randomly fragmented genomic DNA to a planar, optically transparent surface and solid phase amplification to create a high-density sequencing flow cell with millions of clusters, each containing about 1,000 copies of template per sq. cm. These templates are sequenced using four-color DNA sequencing-by-synthesis technology. The MiSEQ™ system uses TruSeq, Illumina's reversible terminator-based sequencing-by-synthesis.

### 2. True Single Molecule Sequencing

A sequencing technique that can be used in the methods of the resent disclosure includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, cleavage removes the fluorescent label, and the process is repeated with other fluorescently labeled nucleotides until a particular read length is achieved. Sequence information is collected with each nucleotide addition.

### 3. 454 Sequencing

Another example of a DNA sequencing technique that can be used in the methods of the present disclosure is 454 sequencing (Roche) (Margulies et al., 2005). 454 sequencing involves two processes. In the first process, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second process, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

### 4. Genome Sequencer FLX™

Another example of a DNA sequencing technique that can be used in the present methods is the Genome Sequencer FLX systems (Roche/454). The Genome Sequences FLX systems (e.g., GS FLX/FLX+, GS Junior) offer more than 1 million high-quality reads per run and read lengths of 400 bases. These systems are ideally suited for de novo sequencing of whole genomes and transcriptomes of any size, metagenomic characterization of complex samples, or resequencing studies.

### 5. SOLID™ Sequencing

Another example of a DNA sequencing technique that can be used in the methods of the present disclosure is SOLID technology (Life Technologies, Inc.). In SOLID sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

### 6. Ion Torrent™ Sequencing

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection-no scanning, no cameras, no light—each nucleotide incorporation is recorded in seconds.

### 7. SOLEXA™ Sequencing

Another example of a sequencing technology that can be used in the methods of the present disclosure is SOLEXA sequencing (Illumina). SOLEXA sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification are repeated.

### 8. SMRT™ Sequencing

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

### 9. Nanopore Sequencing

Another example of a sequencing technique that can be used is nanopore sequencing (Soni and Meller, 2007). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

### B. Analysis of Sequencing

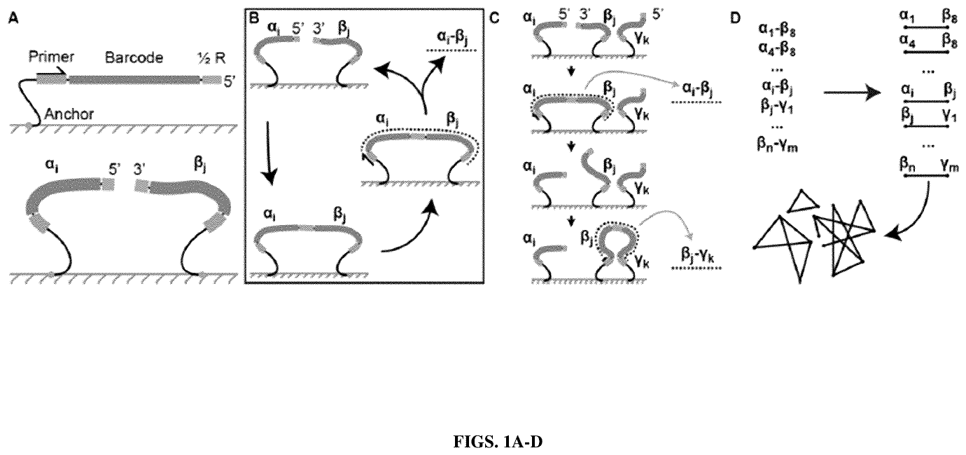

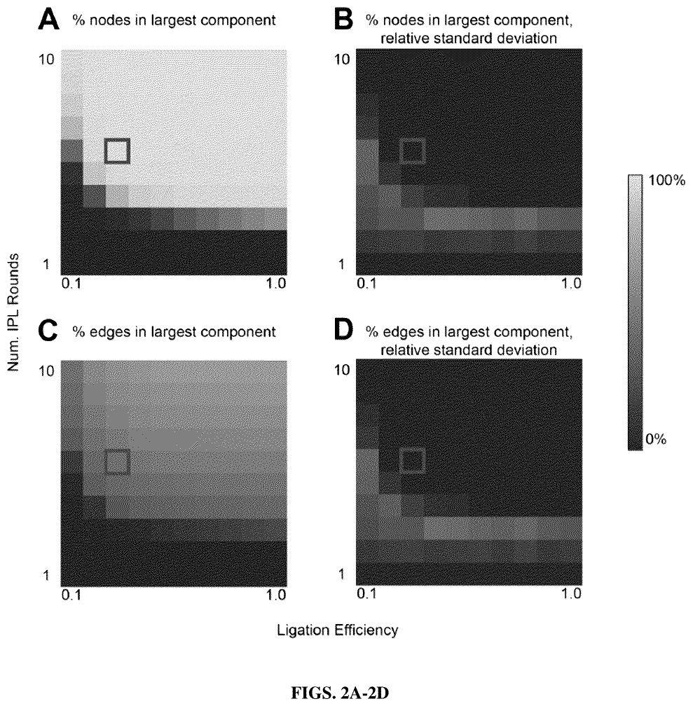

Analysis of the sequencing results may be performed using graph theory, such as for analyzing the IPL output. The readout may be interpreted as a graph wherein the barcoded oligonucleotides are nodes and observed ligations are edges (FIG. 1). The IPL graph may be modeled based on modification of Kama-Kawai algorithm as implemented by Graphviz and networkx, available as open source.

The IPL graphs can be modeled by Erdős-Rényi (ER) random graphs. Briefly, an ER random graph can be constructed by connecting pairs of nodes randomly: each pair may be connected by an edge with probability p independently of all other possible node pairs. If the IPL regime had all oligonucleotides spatially proximal to each other and any oligonucleotide pair has an equal probability of ligating as any other pair during each round, then after multiple rounds each oligonucleotide pair has had an equal and mostly independent chance to ligate. While during one single IPL round ligations between oligonucleotide pairs are mutually exclusive—i.e. each oligonucleotide can ligate to one other oligonucleotide at a time-and hence are not at all independent, over many rounds the cumulative probability of any pair ligating during any round approaches independence.

Random geometric graphs may be used to model the IPL graphs. Briefly, suppose a graph comprises points distributed across a surface with edges connecting points if they are within some distance r from each other. It may be assumed that oligonucleotides were distributed according to a homogenous Poisson point process (Penrose, 2003) within some defined geometrical shape. Such shapes can, for example, be patterned across slide surfaces using photolithography (Pirrung, 2002) or various other methods₁₄. Unlike the case of ER graphs above, connectivity in RG scenarios is naturally contingent on the specific geometry of the shapes involved and therefore it is more difficult to make general guarantees.

### C. Methods of Use

The present methods may be used to detect a single molecule for various applications including research and clinical applications including diagnostics and therapeutics. The diagnostics may be applied to various diseases including cancer and infectious diseases, such as HIV.

The methods may be used as a single-molecule proteomics platform by uniquely tagging each individual amino acid in a protein mixture. The proteomics may be used for agriculture, drug development, diagnostics, and research.

Further applications include the identification of biomarkers relevant to diseases including cancer and infectious diseases. The samples may be from noninvasive procedures such as collection of saliva, blood, or urine as compared to invasive samples, such as biopsies. The proteomics methods may comprise labeling amino acids residues and using the labels to obtain a pattern or stoichiometry of amino acids in successive peptides. This pattern may be searched against the proteome to identify the proteins. The present methods can be used to producing patterns sufficiently reflective of the peptide sequences to allow unique identification of a majority of proteins from a species (e.g. the yeast and human proteomes). Thus, in some embodiments, the present methods may be used for protein or peptide (e.g., hormones) diagnostics, such as from a non-invasive sample including blood, urine or saliva.

Other applications include determining the distribution of receptors on various cell types in order to understand cellular functions and disease states. The methods may be used to detect dimerizing receptors or signaling domains of cellular receptors. The methods may also be applied to other cell types or molecules, such as carbohydrates.

In further applications, the shape of molecules may be determined. For example, nanobeads may be coated with IPL oligonucleotides to recover bead shape and size with high resolution.

### II. EXAMPLES

The following examples are included to demonstrate some embodiments of the disclosure. It can be appreciated that the techniques disclosed in the examples which follow represent techniques discovered by the inventor to function well in the practice of the disclosure. However, those of skill in the art can, in light of the present disclosure, appreciate that many changes can be made in the specific embodiments which are disclosed and still obtain a like or similar result without departing from the spirit and scope of the disclosure.

### Example 1

### Iterative Proximity Ligation

Iterative proximity ligation as a strategy to recover spatial information by DNA sequencing: Iterative proximity ligation (IPL) is based upon the observation that the processes of enzymatic ligation and digestion of DNA are (ideally) memoryless and hence can be applied repeatedly to the same strand multiple times. This observation was used to extend the concept of proximity ligation assays-which are typically limited to one ligation event per oligonucleotide pairs-to allow for multiple ligations per oligonucleotide. Thus, instead of being able to obtain one piece of information, namely that two particular oligonucleotides are in sufficient proximity to each other to ligate, many readouts of proximity information can be obtained about an oligonucleotide and its neighbors (FIG. 1).

These readouts can be interpreted as a graph, where barcoded oligonucleotides are nodes and observed ligations are edges.

In particular, in order for oligonucleotides to have been ligated together, they are in close spatial proximity within a distance bounded by the lengths of the ligated molecules. Thus, the IPL graph intrinsically captures distance relationships among the molecules represented within the graph. Given enough measurements of pair-wise distances, it was shown that it is possible to triangulate and determine the position each molecule is held to generate the observed sequence pairs.

Computational simulations were performed to determine the feasibility for DNA sequencing to determine the spatial relationships among many molecules simultaneously. It was confirmed that there is a complete path from immobilized DNA molecules to sequence pairs and back to the implied positions of the DNA molecules, with positional errors on the order of the oligonucleotide chain lengths. Graph-theoretic properties common to several variants of IPL graphs are discussed, and which can be applied, but it not limited to, to two potential applications: nano-to micro-meter scale microscopy via DNA sequencing and single molecule proteomics.

IPL recovers complete graphs even for very large oligonucleotide pools: random graph theory and simulations: Connectivity across a population of oligonucleotides is the primary readout obtained from IPL: the most important property sought in IPL graphs is for them to be connected because disconnected components are not very mutually informative. It was anticipated that a primary source of error common to all IPL schemes is failure of adjacent oligonucleotides to ligate, which risks fragmenting what in truth are connected oligonucleotide populations into many disconnected graphs. Fortunately, results from random graph theory and simulations showed that disconnected graphs rapidly become extremely unlikely after a handful of IPL rounds.

Two specific random graph models were used. The first model concerned IPL regimes where all oligonucleotides are in mutual proximity to each other and all pairs have an equal chance of ligating during each IPL round, and the second model concerned IPL regimes where the spatial distribution of oligonucleotides restricted each one to ligate its neighbors. These models are readily generalizable: each model's assumptions cover a wide range of potential IPL applications other than those explicitly proposed in the present studies.

The first variant of IPL graphs was well-modeled by Erdős-Rényi (ER) random graphs (Erdos and Renyi, 1964). Briefly, an ER random graph was constructed by connecting pairs of nodes randomly: each pair was connected by an edge with probability p independently of all other possible node pairs. If the IPL regime had all oligonucleotides spatially proximal to each other and any oligonucleotide pair has an equal probability of ligating as any other pair during each round, then after multiple rounds each oligonucleotide pair has had an equal and mostly independent chance to ligate. While during one single IPL round ligations between oligonucleotide pairs are mutually exclusive—i.e. each oligonucleotide can ligate to one other oligonucleotide at a time—and hence are not at all independent, over many rounds the cumulative probability of any pair ligating during any round approaches independence. The IPL scheme additionally deviates from ER assumptions in that its nodes have polarity: 5′ nodes can ligate to 3′ nodes and vice versa. However, if oligonucleotides of each polarity are considered as separate populations whose members are randomly “ligated” to each other after two IPL rounds—the extra round for probes of opposite polarity to act as intermediates-then the methods revert to near-ER assumptions with, at worst, a 2× penalty of rounds required.

IPL rounds were simulated on large graphs with 1000 mutually accessible nodes, with each node randomly chosen to have a 5′ or a 3′ polarity and ligation possible between 5′ and 3′ nodes. The simulations showed that even for such large graphs and with per-oligonucleotide-pair ligation probabilities of 10₋₂ per-round, complete graphs can be recovered with very high probability within five rounds (FIG. 2, top).

Graph theory provided further reassurances that even for large numbers of nodes, connected graphs were nevertheless very likely to be recovered. A result due to Erdős and Rényi₉ is that the connectivity of the ER graph has a threshold or “phase transition” property: if n is the number of nodes in the graph and p=_{clognn} where c is a constant, then as n→∞ the graph will almost certainly be disconnected if c<1 and will almost certainly be connected if c>1. Erdős and Rény provide an additional characterization of ER connectivity thresholding: if p=_{logn+cn} where c is a constant, then as n→∞ the probability of the graph being connected is e−ₑ_{_{−c}}.

A second variant of IPL graphs was well-modeled by random geometric graphs (Penrose, 2003) (RG). The present studies were restricted to oligonucleotides distributed across two-dimensional surfaces, although analogous results exist for higher dimensions. Briefly, suppose a graph comprises points distributed across a surface with edges connecting points if they are within some distance r from each other. In the present scenario, it was assumed that oligonucleotides were distributed according to a homogenous Poisson point process within some defined geometrical shape. Such shapes can, for example, be patterned across slide surfaces using photolithography or various other methods₁₄. Unlike the case of ER graphs above, connectivity in RG scenarios is naturally contingent on the specific geometry of the shapes involved and therefore it is more difficult to make general guarantees. There are additional complications such as edge effects, where nodes near the edge of a geometric distribution will differ in their connection opportunities as compared to their peers well inside the shape, and this effect may be exacerbated in the IPL scenario because polarity mismatch may prevent outlying oligonucleotides from connecting.

However, these concerns are readily obviated if the oligonucleotide density is sufficiently increased (Balister, 2008). Simply put, since each local neighborhood of oligonucleotides is deposited independently of all other neighborhoods, and since at sufficient oligonucleotide density it becomes almost certain that all neighborhoods will become connected (barring some possible outliers), then almost all oligonucleotides within the area of interest belong to the same IPL graph. Although estimates for the required density under various assumptions exist, there is no model that incorporates the oligonucleotide polarity constraint. Therefore, simulations were used to demonstrate that above a certain oligonucleotide density a connected IPL graph containing all nodes can be recovered (FIG. 2, bottom).

### Example 2

### Applications of Iterative Proximity Ligation

Recovery of positional information by DNA sequencing and spring graph layouts: IPL can be used to obtain spatial information about shapes much larger than the molecules used. The present studies demonstrated by simulation that two-dimensional patterns can be recovered by applying spring layout algorithms to IPL graphs.

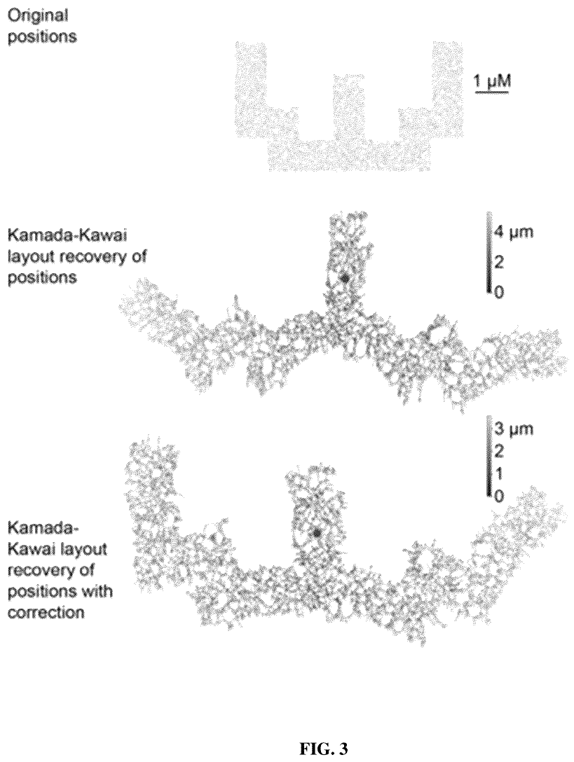

First, deposition of DNA oligonucleotides on a flat glass slide was simulated as a Poisson point process (Kingman, 1993). Each oligonucleotide was randomly assigned a 5′ or 3′ polarity with equal probability. It was assumed that oligonucleotides were covalently attached within a shape (FIG. 3A). It was assumed that after multiple rounds of IPL that any probes sufficiently close to each other would ligate at least once with certainty. The list of barcode pairs thus obtained was used to recover the layout: no additional knowledge whatsoever about oligonucleotide positions was used.

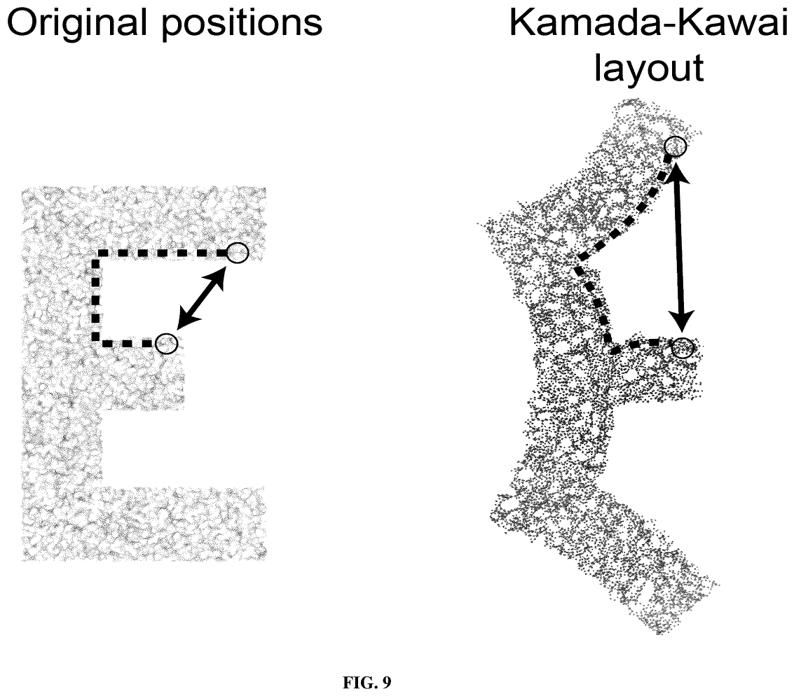

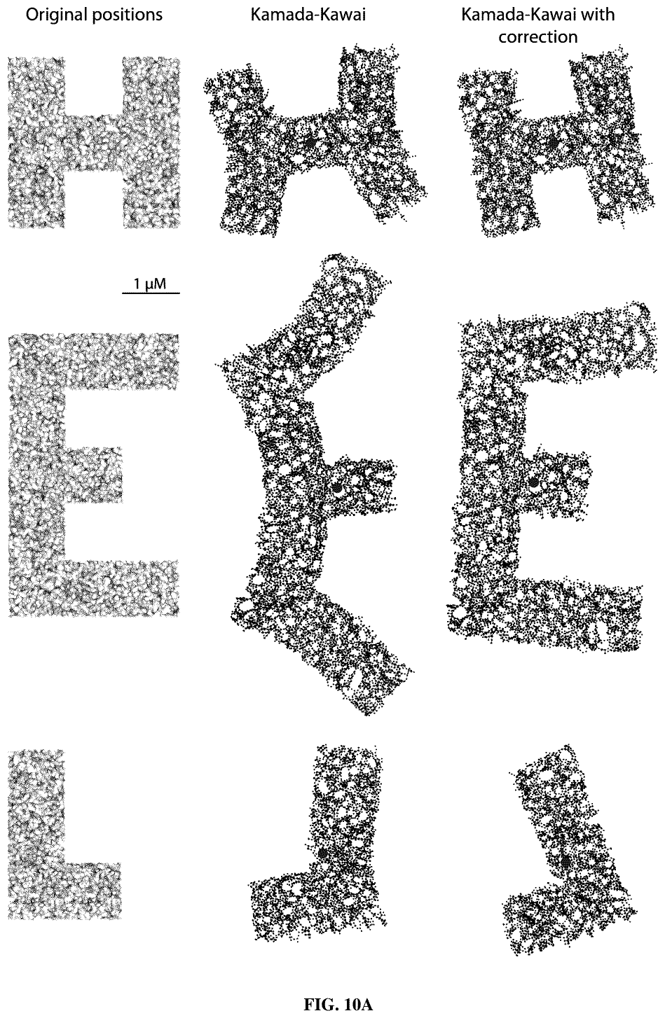

The Graphviz Neato implementation of the Kamada-Kawai (KK) algorithm (Kamada and Kawai, 1989) was used to recover the layout (FIG. 3B). The algorithm initializes node positions randomly and thus may cause topologically twisted layouts (FIG. 9), so a layered layout strategy was pursued. The graph center—defined as the set of nodes with graph eccentricity equal to graph radius (Harary, 2001)—was identified and a randomly selected central node was used as a seed to layout the neighborhood of radius 1 around it. The positions of nodes in this layout were used to initialize the layout the neighborhood of radius 2. This cycle was repeated until all nodes were laid out. The recovered shape was scaled such that the median distance between oligonucleotides in the recovered layout was equal to the median distance in the original shape. Note that due to the nature of the Poisson point process this median value is, except in pathological cases, dependent on the Poisson parameter 2 and hence highly conserved between different shapes. To measure large scale shape distortion in the layout versus the actual oligonucleotide positions, the original shape and the recovered layout were aligned. First, the node in the closest to the centroid of the actual shape was chosen as a coordinate origin. The same node (red spot in FIG. 3B) in the layout was aligned to the coordinate origin, and the optimal rotation (within 0.1° resolution) that minimizes the sum of Euclidean distances between nodes' original and recovered positions was found. For each node, the Euclidean distance was measured between its original and recovered positions (FIG. 3B, heatmap).

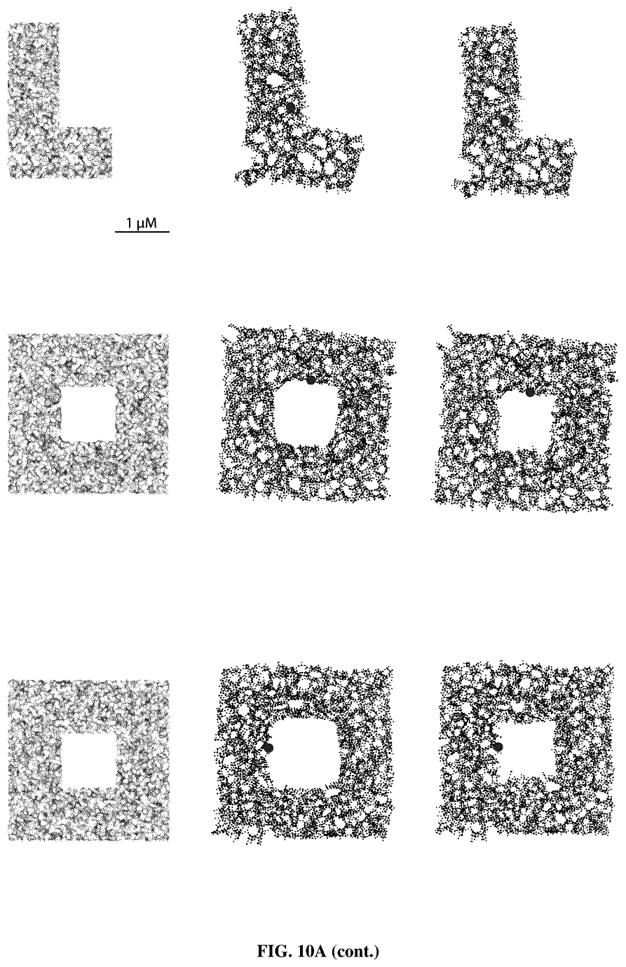

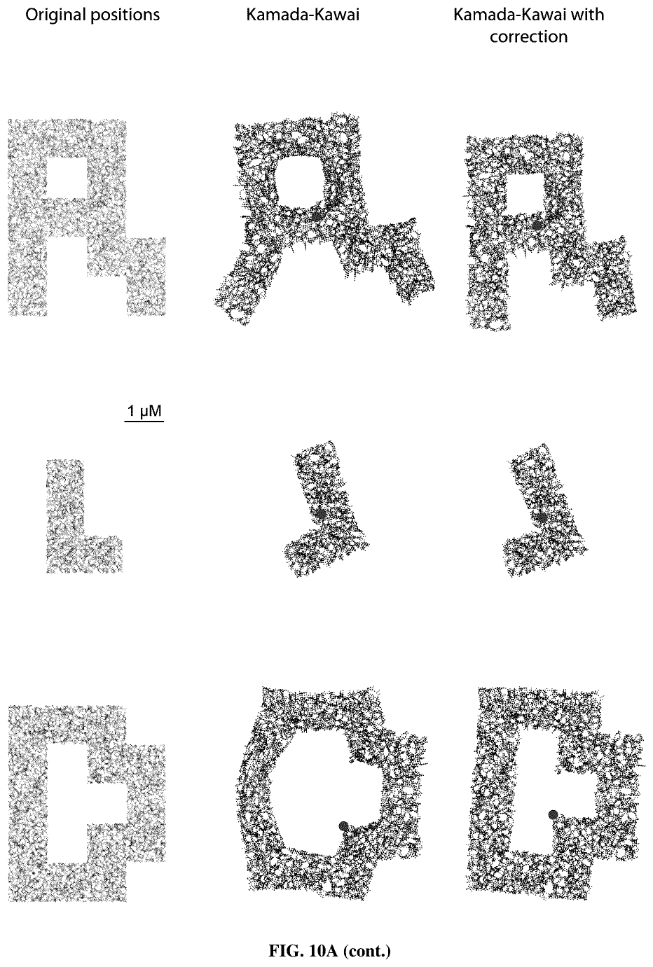

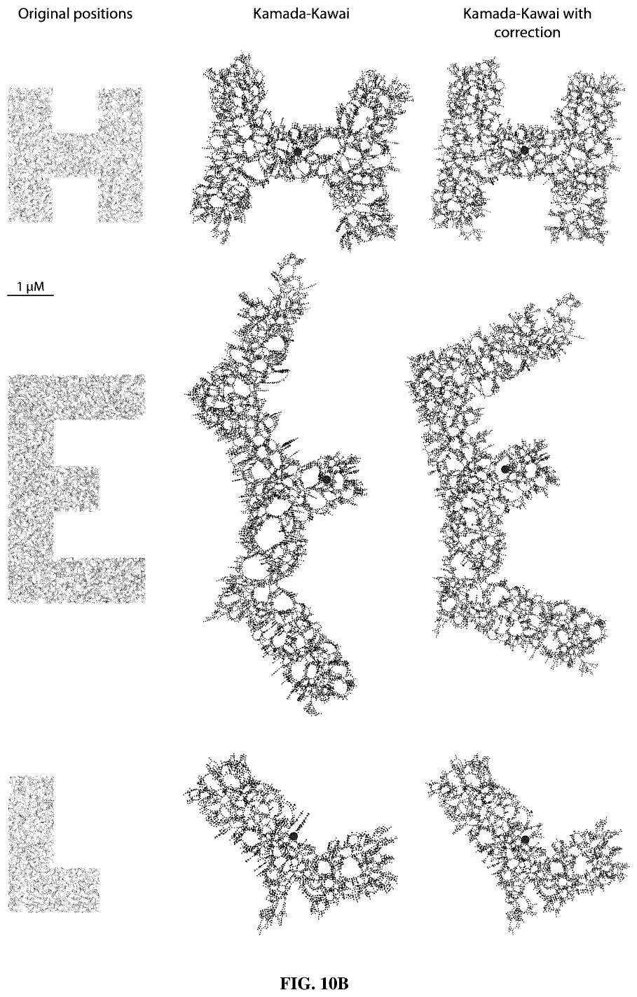

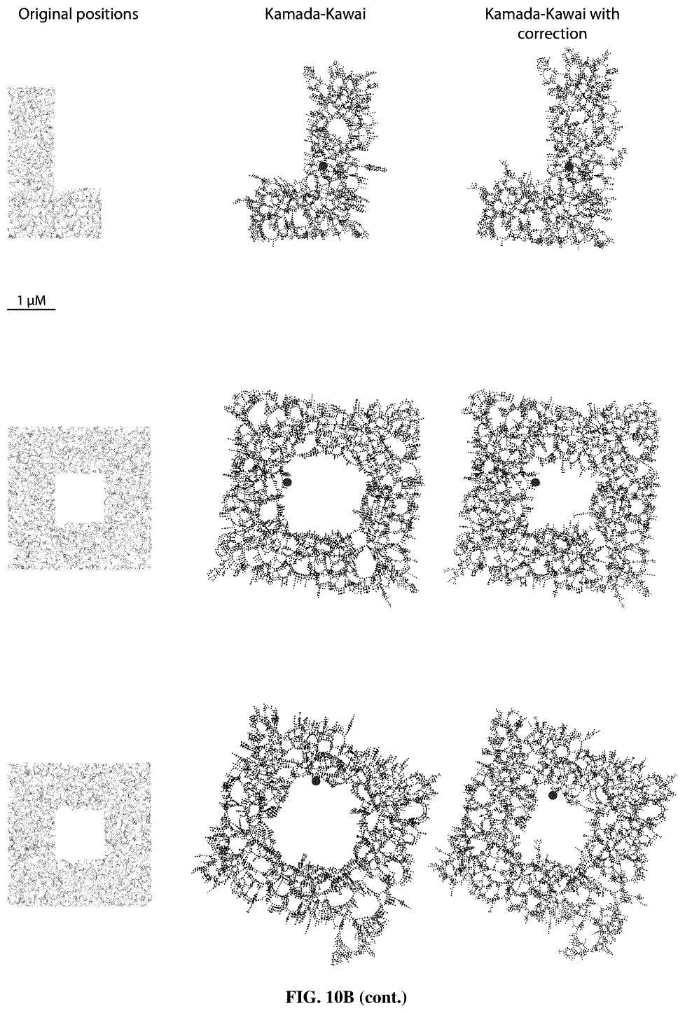

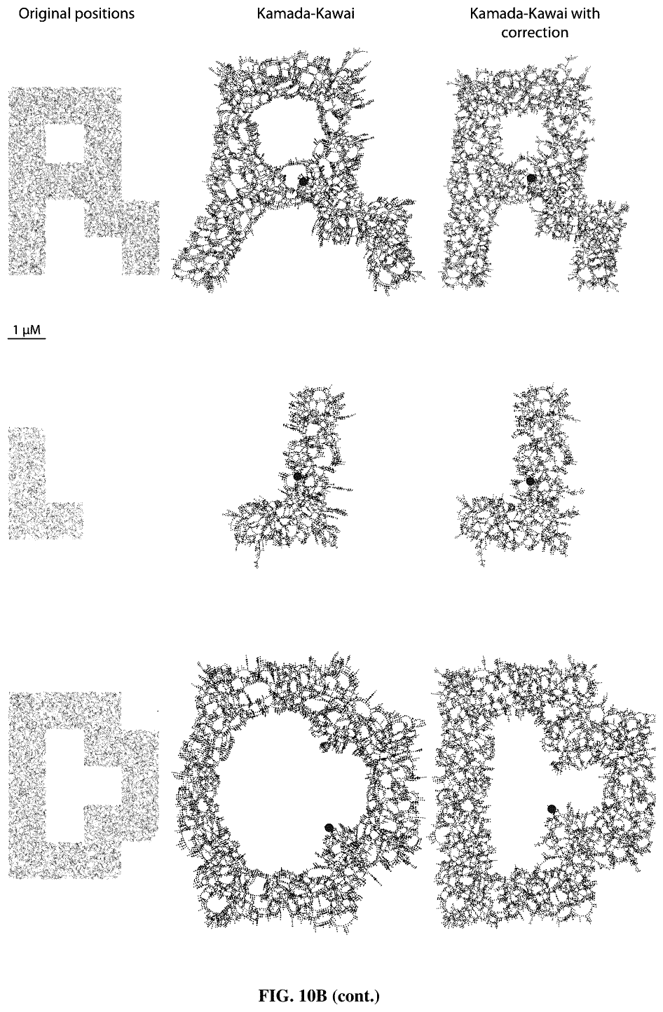

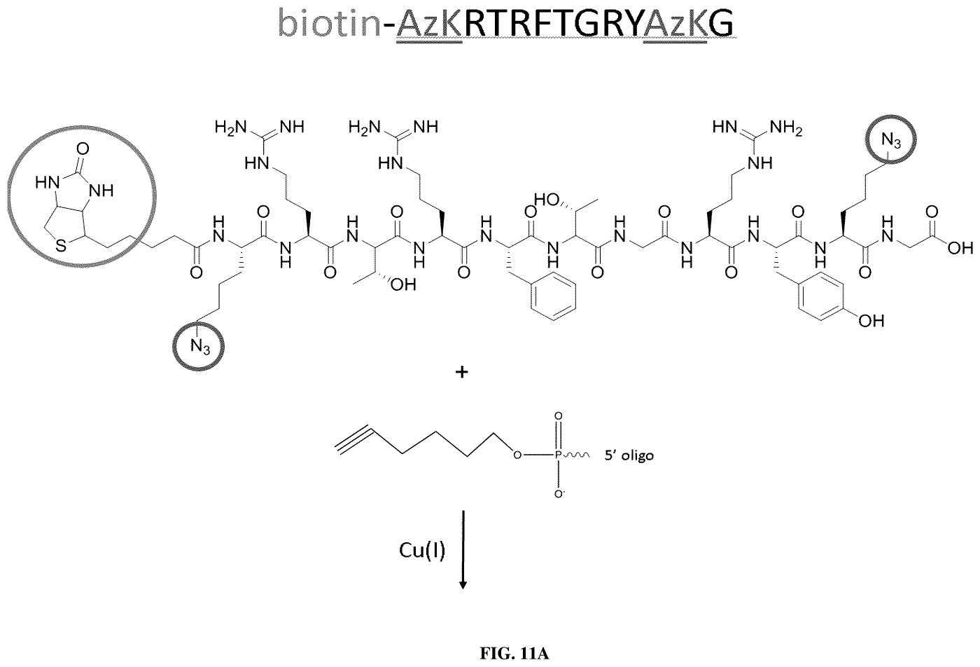

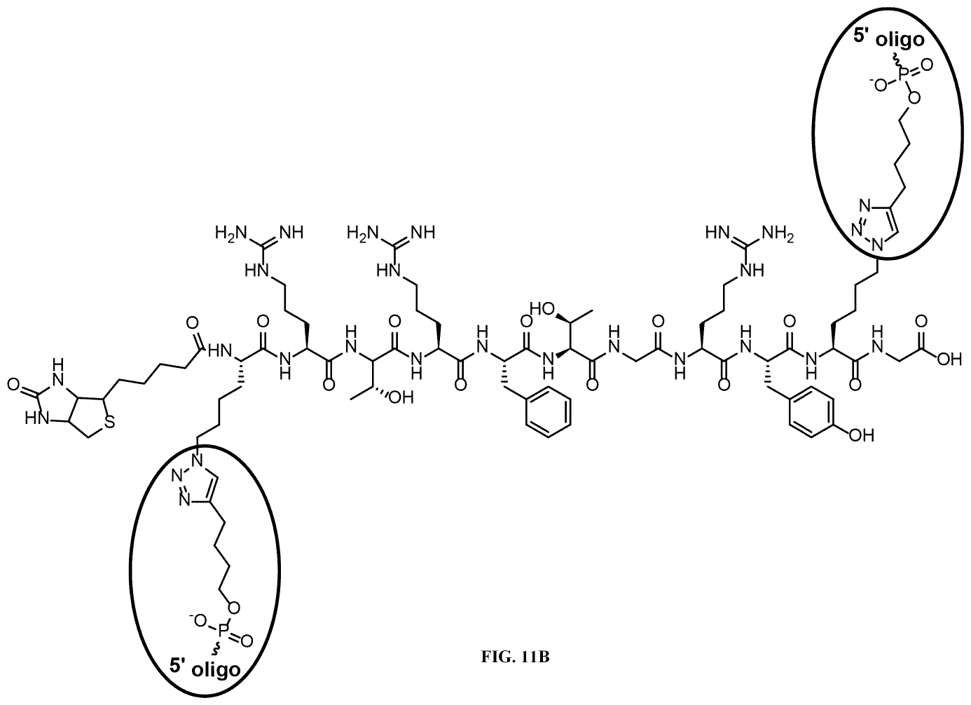

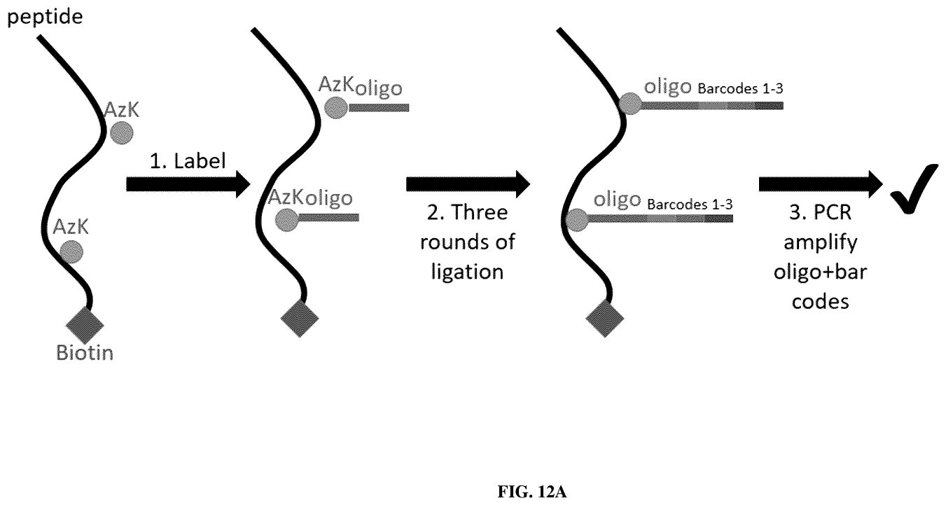

The primary cause of distortion was the discrepancy between the fundamental assumptions of the KK algorithm and concave geometries (FIG. 10). To correct for this, the recovered layout was taken and re-fed into a modified variant of the KK algorithm that performs local-only spring energy minimization instead of a global minimization. This significantly decreased large-scale distortions.

Using this approach, all the letters for message “HELLO WORLD” (FIG. 11) were recovered. Note that the orientation of some letters is inverted and that this is equally consistent with the information provided: this ambiguity cannot be resolved without additional information such as fiduciary markers or connections spanning the letters.

KK layout of two-dimensional patterns recovered not only large-scale structure (FIG. 3C) but also finer structural details (FIG. 3A′-C′). To quantify layout fidelity at smaller scales, 50 nodes (oligonucleotides) were randomly sampled from each letter in “HELLO WORLD” and their neighborhoods of graph radius 10 were examined, corresponding to a physical distance of approx 597 +/−82 nm (mean +/−std.dev.) from the sampled node. For each oligonucleotide in this neighborhood, the distance between its original and recovered position was compared with the sampled node acting as the common coordinate origin for the local comparison. The difference in position for all nodes was 48 +/−33 nm with a median of 42 nm. It was expected that nodes further on the graph from sampled node would have a larger error: looking at nodes 10 hops from the sampled node, the difference in position was 52 +/36 nm with a median of 46 nm. The recovered oligonucleotide positions within a half-micrometer radius circle deviate from their true positions by 5% of its diameter. Note that each oligonucleotide is approx. 34 nm in length, meaning that the average error in recovered position is smaller than the maximum reach of an oligonucleotide pair. As can be observed around the “holes” in FIG. 3A′-C′, a primary cause of local distortion is the same concave geometry problem above that affects larger-scale structures.

Single-molecule proteomics: By labeling proteins with oligonucleotides, IPL can be applied as a method for single-molecule proteomics. It was demonstrated by simulation that the vast majority of a single cell's proteome can be reliably detected and quantified using this technique.

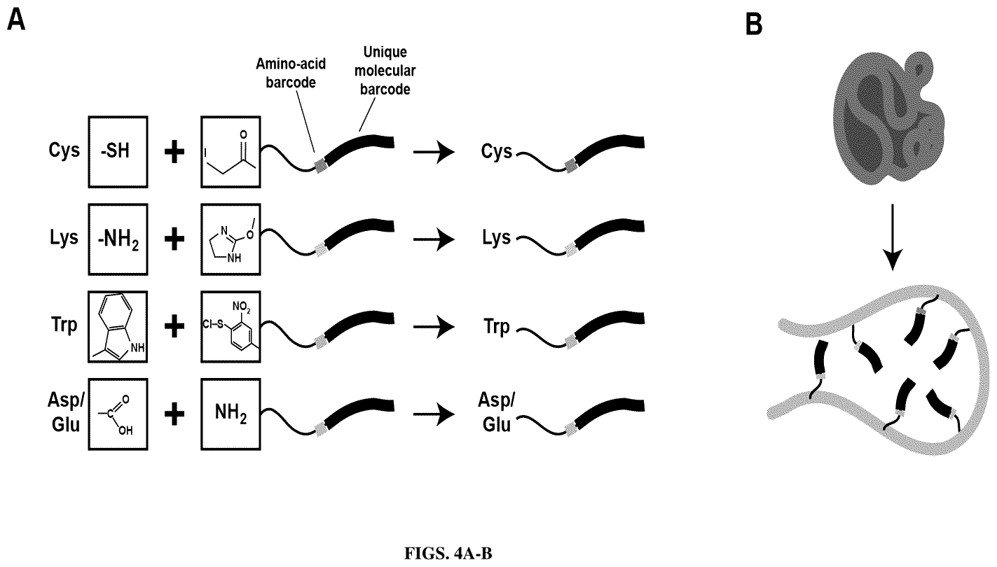

A protein labeling scheme (Hernandez, 2017) was used for the single-molecule peptide sequencing technology (Swaninathan, 2015). This scheme was adapted to selectively tag amino acids with a variant of IPL oligonucleotides such that each oligonucleotide encodes the identity of its target amino acid species and a molecular barcode unique to the individual amino acid even amongst a large pool of proteins (FIG. 4). Once proteins are labeled, they are diluted and IPL is applied to the entire solution in bulk. If the proteins are sufficiently dilute, then oligonucleotides on the same protein will ligate and yield intramolecular oligonucleotide pairs while oligonucleotides on separate proteins will be extremely unlikely to ligate and hence will not yield intermolecular pairs. Since the proteins are denatured upon labeling, all oligonucleotide pairs on a single protein are equally likely to ligate and therefore, per the graph-theoretical considerations above, the protein's IPL graph is well-modeled as an Erdős-Rényi random graph with extremely likely connectivity after several rounds. Each protein will thus yield its own connected IPL graph, with nodes representing its labeled amino acids. Tallying amino acids from the graph recovers the protein's partial compositional information, which is sufficient to identify a substantial proportion of a proteome (FIG. 5A).

Protein identification is significantly boosted if, after this initial sequence of IPL rounds, the proteins are site-specifically digested via e.g. trypsin and a second sequence of IPL rounds is applied to the digested peptides. Since each amino-acid's barcode is unique in the entire population, they can be used to reconcile barcode pairs obtained from both rounds. The additional information from the second stage yields compositional information about each fragment, and the specificity of the proteolytic enzyme contributes partial sequence information. This additional information allows even two-label schemes to resolve the vast majority of proteins in a proteome (FIG. 5B).

Labeling efficiencies for cysteines and carboxylates are quantitative (Schnatbaum et al., 2016). Nevertheless, to characterize the method's robustness against labeling failure, simulated protein identification was simulated under progressively lower labeling efficiencies. This approach is analogous to strategies for computationally evaluating the feasibility the single-molecule protein sequencing technology. Briefly, 10⁴ copies of each protein in the E. coli proteome were simulated to undergo labeling at their Cys and/or Asp/Glu residues at a given stochastic probability, with each amino-acid labeling event modeled as an independent Bernoulli random variable. It was assumed that all proteins yielded complete IPL graphs incorporating all labeled amino acids, and that a two-stage strategy yielded amino acid compositions of all tryptic peptides in each protein. Thus, the combination of amino acid compositions for the tryptic peptides was obtained as the compositional signature of each protein. A protein was considered identifiable if it yielded a compositional signature at least 10 times (out of 10⁴), with all other proteins contributing at most 10% of that signature's total occurrences. The results showed that even under a 90% efficient labeling regime, the two-stage IPL strategy can still identify a substantial portion of the proteome (FIG. 5C).

Initial Instantiation of iterative proximity ligation (IPL): In order to demonstrate the feasibility of iterative proximity ligation (IPL), streptavidin was used to immobilize biotinylated oligonucleotides adjacent to one another. The oligonucleotides were designed such that their ligation can be reversed by the addition of a restriction endonuclease, leading to potential rounds of ligation, cleavage, and re-ligation (Table 1). One such round was performed, using qPCR to quantify the proportion of ligated products after each reaction (FIG. 6). Six replicates of these experiments were performed.

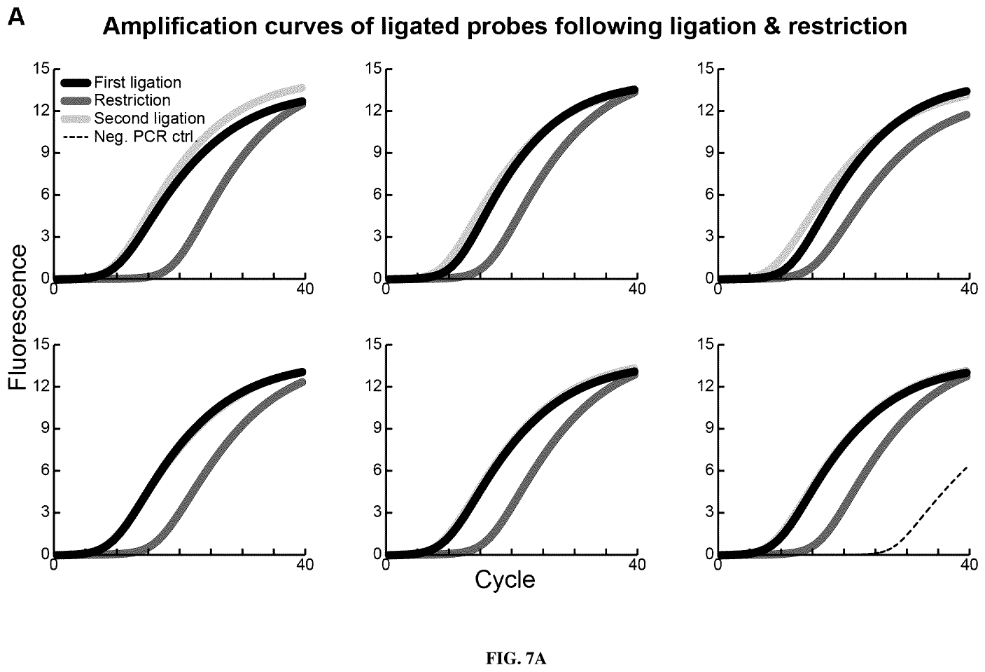

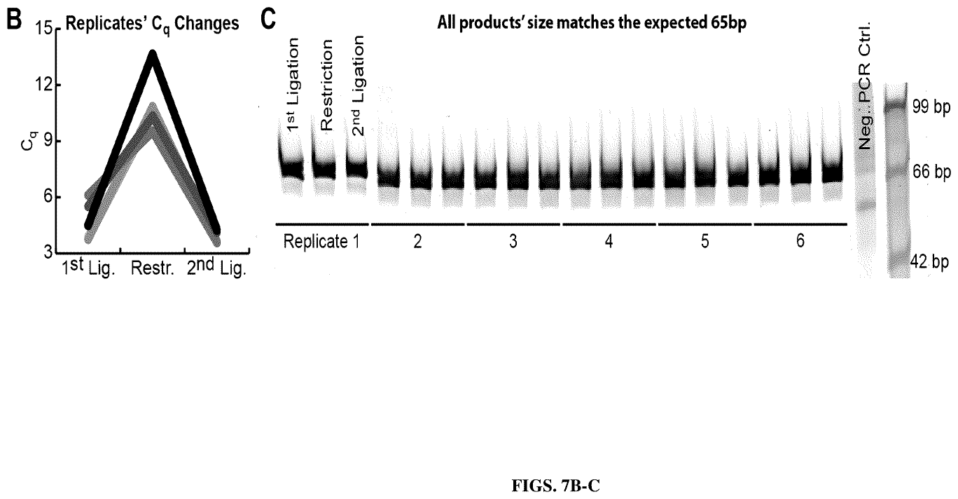

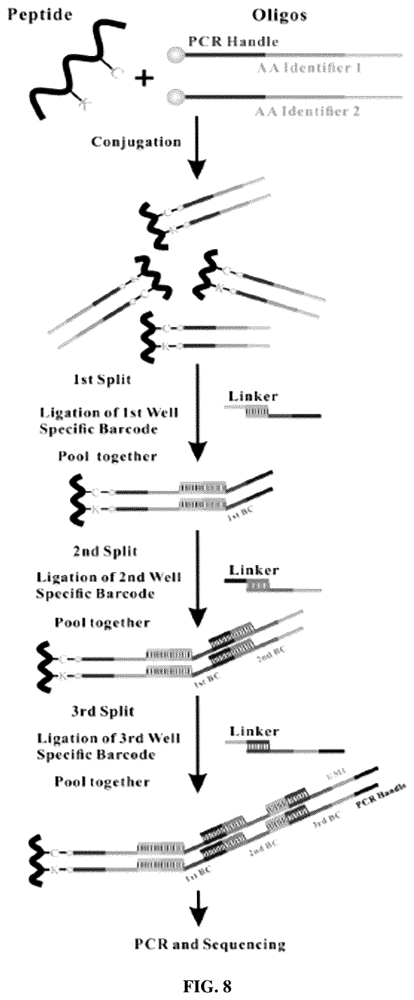

Initially, biotinylated left probe and right probe (2.5 uL of 10 uM stocks, each) were mixed and incubated with 2 uL streptavidin beads in 1× B&W buffer, with the probe and bead concentrations adjusted to ensure there were approximately equimolar numbers of probes and streptavidin binding sites. Upon the addition of T4 ligase and 1 mM ATP and further incubation for 5 min at 37° C., a 10 uL sample was taken for qPCR, which yielded an average C_{q} (time to signal) of 4.6±1.0 (mean±std. dev. across six replicates) (FIG. 7B). The beads were then washed and incubated with EcoRV-HF enzyme for 2 hours at 37° C. to cleave templates that yielded amplicons, and another 10 uL sample (equivalent in concentration to the first) was taken for qPCR analysis. The average increase in C_{q} for the digested samples was 6.3±2.0, indicating that a significant portion of the ligated probes had indeed been digested. To regenerate the template, the beads were again washed to remove the restriction endonuclease and then incubated with a more concentrated T4 ligase for 15 min at 37° C. The 10 uL sample showed a decrease in C_{q} of 7.1±1.2, indicating that cleaved probes were being religated (FIGS. 7A, B). Overall, the C_{q} values went from 4.6±1.0 to 10.9±1.4 upon cleavage to 3.8±0.31 upon religation. All qPCR products were confirmed to correspond to the correct size as expected from the designs (Table 1) by 10% PAGE (FIG. 7C).

TABLE 1
Oligonucleotide sequences.

| Oligo- | | SEQ |
|---|---|---|
| nucleo- | | ID |
| tides | Sequences | NO: |
| left | /5Biosg/TT TTT TTT TTT | 1 |
| probe | TTT TTT TTT TTT TTT TTT | |
| | TTT TTT TTT TTT TTT TTT | |
| | TTT TTT TTT TTT/iSp18/ | |
| | CGC TTC AGG TAG TAG TAC | |
| | GTC TAT GTA TGA T | |
| right | /5Phos/ATC CTG TAG CAT | 2 |
| probe | TAA TAC TCT CGC AGC ACA | |
| | ACG GTA C /iSp18/ TTT | |
| | TTT TTT TTT TTT TTT TTT | |
| | TTTTTT TTT TTT TTT TTT | |
| | TTT TTT TTT TTT TT /3Bio/ | |
| connector | CTA CAG GAT A TCA TAC AT/ | 3 |
| | 3InvdT/ | |
| forward | CGC TTC AGG TAG TAG TAC GTC T | 4 |
| primer | | |
| reverse | C CGT TGT GCT GCG AGA GTA TTA | 5 |
| primer | | |

### Example 3

### Split-Seq Single Molecule Proteomics

In addition to using IPL to identify amino acid-specific amplicons within the same protein, a separate method was proposed for barcoding and identifying amino acids within a protein.

As with IPL, oligonucleotides with both an amino acid identifier and a unique identifier will be conjugated to individual amino acids within individual proteins. The sample is then split into a number of wells (i.e., in a 96-well plate) and ligated to an additional, well-specific barcode to the all of the conjugated oligonucleotides. The samples are then pooled, and re-allotted to different wells, and a second, well-specific barcode is appended; this diversifies the number of different barcode combinations on individual samples to ca. 96×96˜10,000 (for example). Additional rounds of pooling, splitting, and ligation are carried out in order to further diversify the combinatorial, ligated barcodes to the point where there is a unique combinatorial, ligated barcode for each protein or peptide. The key result is that each protein has its own unique barcode, and a copy of this barcode has been appended to each of the protein's labeled residues.

For example, a single protein or peptide that contained two cysteine residues and a lysine residue might have the following sequences appended to it, after three rounds of split-and-pool ligation:
Cysteine identifier—unique barcode—well code 1—well code 2—well code 3
Cysteine identifier—unique barcode—well code 1—well code 2—well code 3
Lysine identifier—unique barcode—well code 1—well code 2—well code 3

In contrast, a different single protein with two lysines and one cysteine might have the following sequences appended to it:
Cysteine identifier—unique barcode—well code 1—well code 4—well code 5
Lysine identifier—unique barcode—well code 1—well code 4—well code 5
Lysine identifier—unique barcode—well code 1—well code 4—well code 5

As can be seen, reading out the well codes distinguishes the two proteins, and thus also indicates which residues go together on these proteins. The individual residues on each protein are distinguished by their “unique barcode” sequences (italicized), and hence the number of each kind of amino acid (i.e., cysteine or lysine) that protein contains can be counted. This yields, in effect, the same compositional information as provided by IPL.

Note that the IPL simulations demonstrating that the vast majority of a proteome can be resolved even under suboptimal labeling efficiencies (below those observed in practice) are directly applicable to this technique.

Using this method, upwards of 10¹² proteins can be barcoded in a mixture. This would require up to six rounds of split-and-pool barcode addition for a 96 well plate. There would be potentially fewer rounds for identification depending on the number of well codes used: for example, 5 rounds in a 384 well plate can yield about 10¹¹ unique protein tags. Such numbers are within the grasp of current NextGen DNA sequencing methods, effectively converting single molecule protein composition identification to NextGen DNA sequencing. As more amino acid tags are used, the increasingly precise composition of individual proteins allow their identification (e.g., via look-up tables for organisms).

### Example 4

### Materials and Methods

Simulating Erdős-Rényi (ER) random graph connectivity: IPL ligation was simulated for graphs with 1000 nodes at varying ligation probabilities. Each node was randomly chosen to have a 5′ or 3′ polarity, with ligation possible between opposite polarities. During each round, all possible oligonucleotide pairs were iterated through in random order and each was potentially ligated at the given probability. Each oligonucleotide can ligate to one partner per round: if an oligonucleotide pair successfully ligated at any point during the iteration, its members can not participate in any subsequent pairings during that round. 100 simulation replicates were performed for each ligation probability and the size of the largest component was measured after each round.

Simulating random geometric graph (RG) connectivity: 34 nm long (˜100 base) IPL oligonucleotides were deposited by simulation onto a square pattern on a flat surface (68 nm×15) ₂=1.04 um₂ in size as a Poisson point process with intensity parameter λ=5 oligonucleotides/(68 nm×68 nm). Each oligonucleotide was randomly assigned 3′ or 5′ polarity. During each round, all oligonucleotide pairs within 2×34 nm=68 nm of each other and of complimentary polarities were iterated through in random order and each was potentially ligated at the given probability. Each oligonucleotide can at most participate in one ligation per round. 100 simulation replicates were performed for each ligation probability and the size of the largest component was measured each round. Since a new graph was generated for each ligation probability and their total node number varies, the size of the largest component was expressed as the percentage of the graph's total nodes belonging to it.

Simulating recovery of spatial information (letter patterns) from DNA sequences using spring-layout algorithms: 34 nm long (˜100 base) IPL oligonucleotides were deposited by simulation onto letter shape patterns on a flat surface as a Poisson point process with intensity parameter λ=5 oligonucleotides/(68 nm×68 nm). Each oligonucleotide was randomly assigned 3′ or 5′ polarity. It was assumed all oligonucleotides within 2×34 nm=68 nm of each other and of complimentary polarities were ligated at some point during at least one IPL round.

Representing each IPL oligonucleotide as a node and each ligated pair as an edge, a graph was constructed using the possible oligo-oligo ligations obtained above. Any nodes not belonging to the largest connected component were discarded; the largest connected component was retained for further analysis. In practice, this meant <1% of nodes were discarded.

Graphviz's implementation of the Kamada-Kawai (KK) algorithm was iteratively applied to recover the original letter shape. The graph center—defined as the set of nodes with graph eccentricity equal to graph radius—was identified and used a randomly selected central node as a seed to layout the neighborhood of radius 1 around it. The positions of nodes in this layout were then used to initialize the layout the neighborhood of radius 2. This cycle was repeated until all nodes were laid out. The recovered shape was scaled such that the median distance between oligonucleotides in the recovered layout was equal to the median distance in the original shape.

To obtain a corrected KK layout, networkx's Python implementation²⁷ of the KK algorithm was implemented to ignore node-node spring interactions outside of graph radius 5. The layout obtained from this round of the iterative Graphviz KK layout was fed into this modified function to obtain the corrected layout.

For both corrected and uncorrected variants, the node coordinates were scaled such that the median node-node Euclidean distance in the layout was equal to the median oligonucleotide-oligonucleotide distance between Poisson deposited oligonucleotides.

To characterize distortion of the recovered layout, the node in the original layout closest to the centroid of all oligonucleotide positions was selected as an anchor node. The anchor node in original and recovered layouts were aligned, and rotated the recovered layout (at 0.1° resolution) around it to minimize the sum of Euclidean distances between original and recovered node positions. To account for the recovered shape possibly being a mirror inversion of the original, this was repeated for both orientations, and selected the orientation that minimized this sum. Once the best-fitting alignment orientation and angle were chosen, each node the Euclidean distance was then calculated between its original and recovered position.

Simulating single-molecule proteome coverage: UniProtKB/Swiss-Prot complete E. coli and H. sapiens proteomes (manually reviewed) were downloaded on 16th May 2016 and used for all simulations, comprising 4297 and 19988 annotated proteins, respectively. Alternatively-spliced isoforms were ignored.

Identification of proteins without trypsinization at perfect labeling efficiency: For each labeling scheme, the number of labeled amino acids in each protein were tallied and represented this result as an ordered tuple. For example, a protein with 3 lysines and 5 cystines would be represented as (3, 5). A protein was unique if no other protein's composition was represented by the same tuple.

Identification of proteins using trypsinization at perfect labeling efficiency: Each protein's sequence was cut after each lysine and arginine. Each protein fragment's amino acid composition was represented as a tuple, and each protein's composition was represented as the multiset of fragment tuples. A protein was unique if no other protein's composition was represented by the same multiset of tuples.

Identification of Proteins Using Trypsinization at Sub-Optimal Labeling efficiencies: 10⁴ molecular replicates were simulated for each protein in a Monte Carlo fashion. Each protein's sequence was cut after each lysine and arginine. For each replicate, each amino-acid was labeled with the given stochastic labeling probability. Each individual replicate's composition was represented by a multiset as above, however counting successfully labeled amino acids. For each protein, observed multisets were collated from all 10⁴ replicates. A protein was unique if it had at least one multiset that occurred at least 10 times, and that the number of instances of that multiset observed in all other proteins accounted for at most 10% of all its occurrences across the proteome.

### Iterative Proximity Ligation on Magnetic Streptavidin Beads

Materials and bead preparation: 2× binding and washing (B&W) buffer was prepared per Invitrogen as 10 mM Tris-HCl (pH 7.5), 1 mM EDTA, 2 M NaCl. Biotinylated left and right probes (10 uM; Table 1) were mixed in 1× B&W buffer in a total volume of 30 uL to concentrations of 0.78 uM each. The mixture was incubated with 2 uL of stock Invitrogen Dynabeads M-270 Streptavidin (Invitrogen catalog #65305) at room temperature for 20 minutes to allow immobilization. After binding, beads were washed with 200 uL of 1× B&W buffer and resuspended in 200 uL nuclease-free H₂O.

Iterative proximity ligation: Some 22 uL of resuspended beads was combined with 22 uL of 100 nM connector (Table 1). Ligation was carried out by adding 5 uL 10× ligation buffer (NEB B0202S) and 1 uL of T4 ligase (NEB M0202S) diluted in 1× ligation buffer to 40 U/uL, and incubating for 5 minutes at 37° C.

After ligation, beads were washed using 200 uL of 1× B&W buffer and resuspended in 50 uL of nuclease-free H₂O. 10 uL of resuspended beads were aliquoted for qPCR and stored at 4° C.

The remaining 40 uL of resuspended beads was subjected to digestion. 1 uL of 10 uM connector, 5 uL 10× CutSmart Buffer (NEB B2704S), and 4 uL EcoRV-HF was added to the beads and incubated at 37° C. for 2 hours.

Reactions were washed using 200 uL of 1× B&W buffer and resuspended in 40 uL of nuclease-free H₂O. 10 uL of resuspended beads were aliquoted for qPCR and stored at 4° C.

Ligation was repeated on the remaining 30 uL of beads as above, except using 14 uL of 100 nM connector, 1 uL of 400 U/uL ligase, and incubating at 37° C. for 15 minutes.

After ligation, beads were washed using 200 uL of 1× B&W buffer and resuspended in 30 uL of nuclease-free H₂O. 10 uL of resuspended beads were aliquoted for qPCR and stored at 4° C.

qPCR from magnetic beads: qPCR of all aliquots was performed simultaneously. 3 uL from each 10 uL aliquot was combined with 3.6 uL of nuclease-free H₂O, 1.2 uL of forward and reverse primers (each, 10 uM; Table 1), 1 uL Evagreen dye (Biotium #31000), and 10 uL 2× FastStart DNA Probes Essential Master Mix (Roche #06 402 682 001).

qPCR started with an initial enzyme activation of 95° C. for 600 sec; followed by 40 cycles of 95° C. for 10 sec melting, 62° C. for 1 sec annealing, and 72° C. for 1 sec extension; the three processes to obtain Evagreen melting curves were at 95° C. for 10 sec, 60° C. for 60 sec, and 97° C. for 1 sec.

Gel electrophoresis of qPCR reactions: A 10% polyacrylamide gel was made by combining 12.5 mL 20% acrylamide (in 7 M urea), 12.5 mL TBE dilution buffer (in 7 M urea), 100 uL 10% APS, and 25 uL TEMED. 20 uL of each qPCR reaction was mixed thoroughly with 20 uL 2× loading dye (95% formamide, 10 mM EDTA, 0.025% BB) and denatured at 95° C. for 5 minutes. After denaturation, the temperature was ramped down to 25° C. at the rate of 0.1° C./s. The denatured product was loaded into the gel and run at the voltage of 400 V for 2 h. After running, the gel was stained in 10 uL of 10000X SybrGold (Invitrogen, S11494) diluted with 100 mL H₂O. The oligonucleotide fragments from Bhadra and Ellington (Bhadra and Ellington, 2014) were used as the DNA ladder.

### Example 5

### Split-and-Pool Method

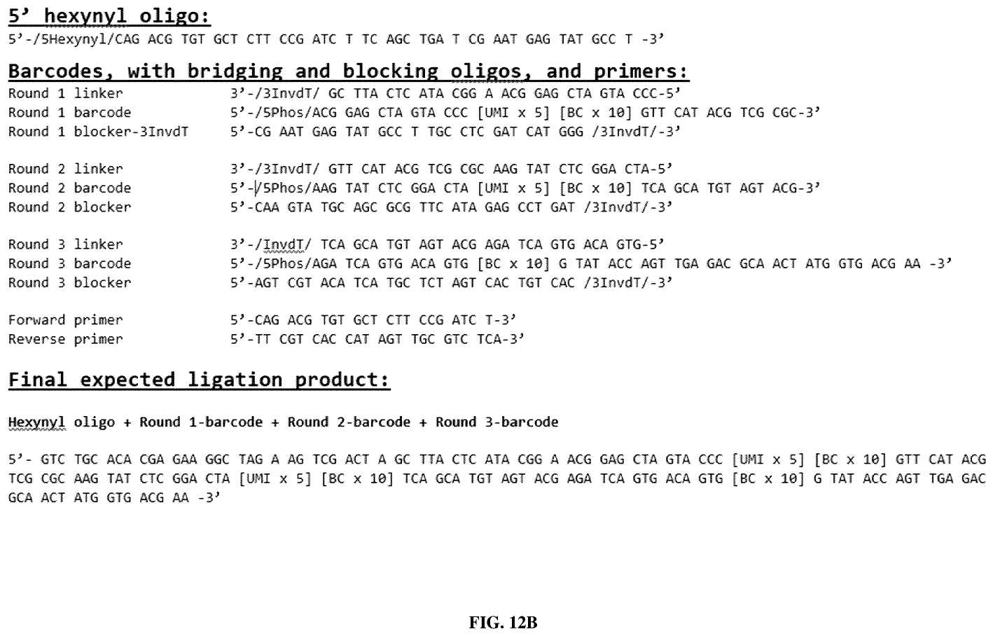

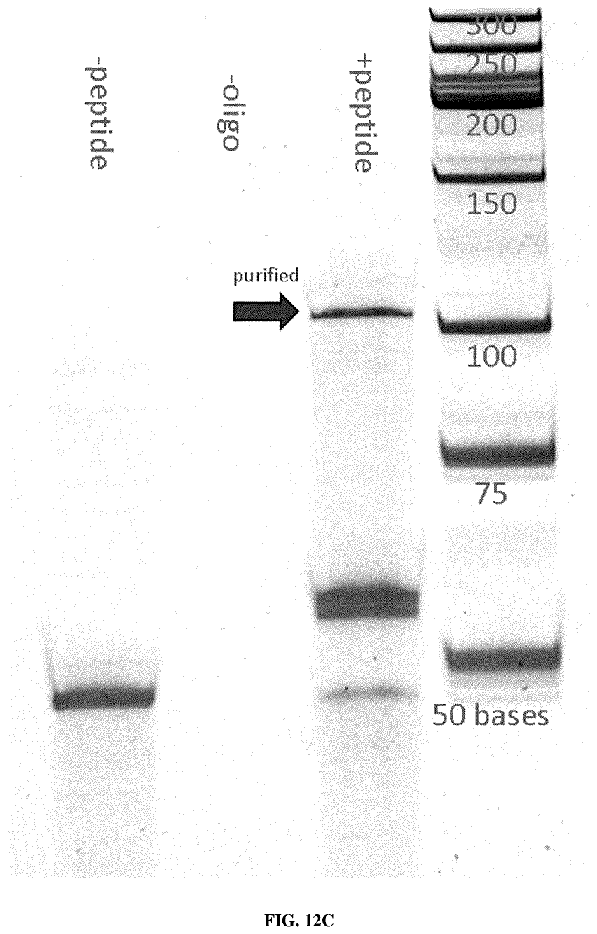

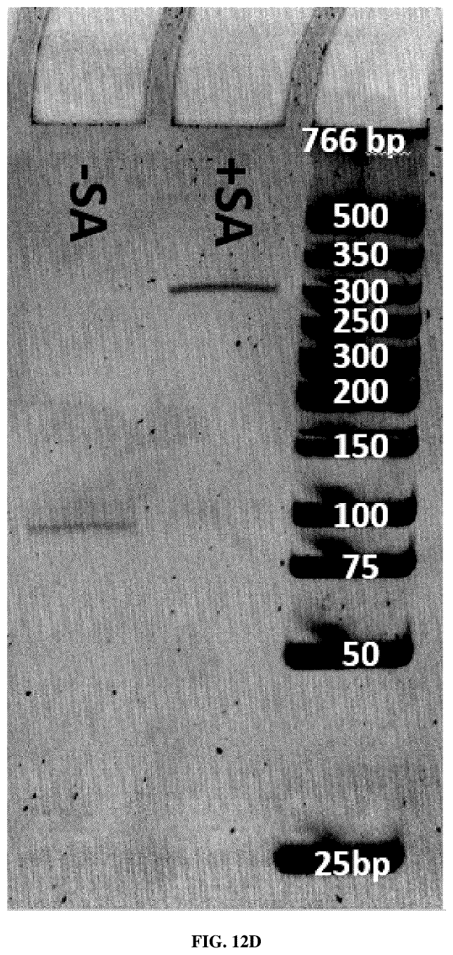

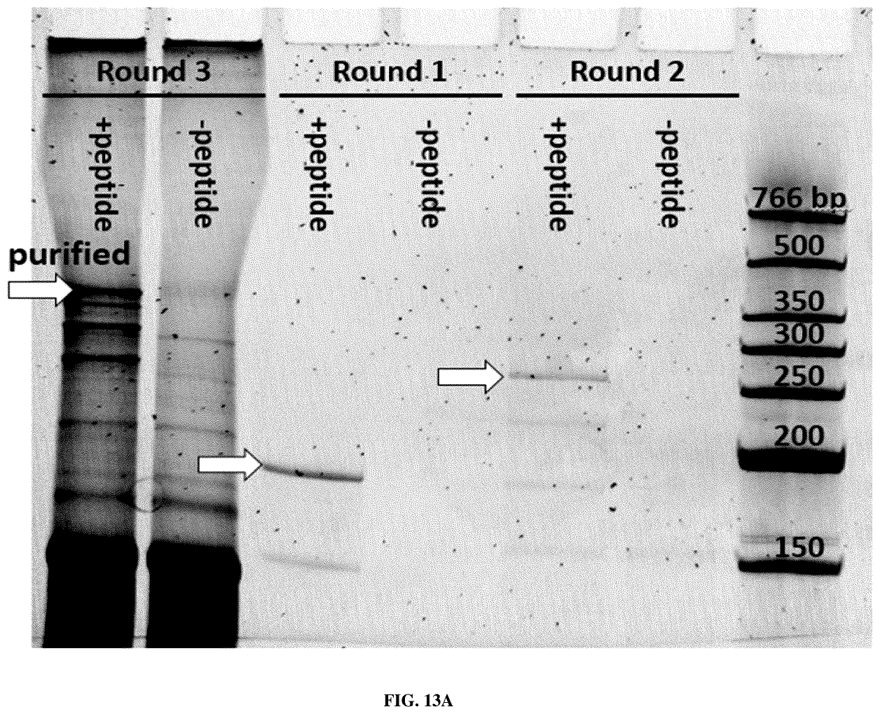

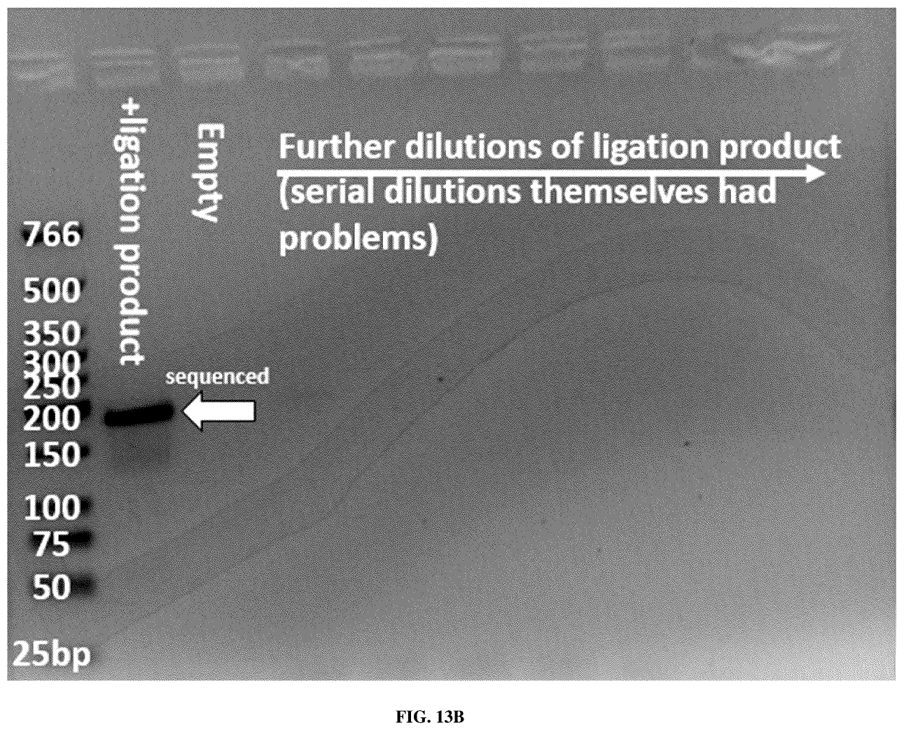

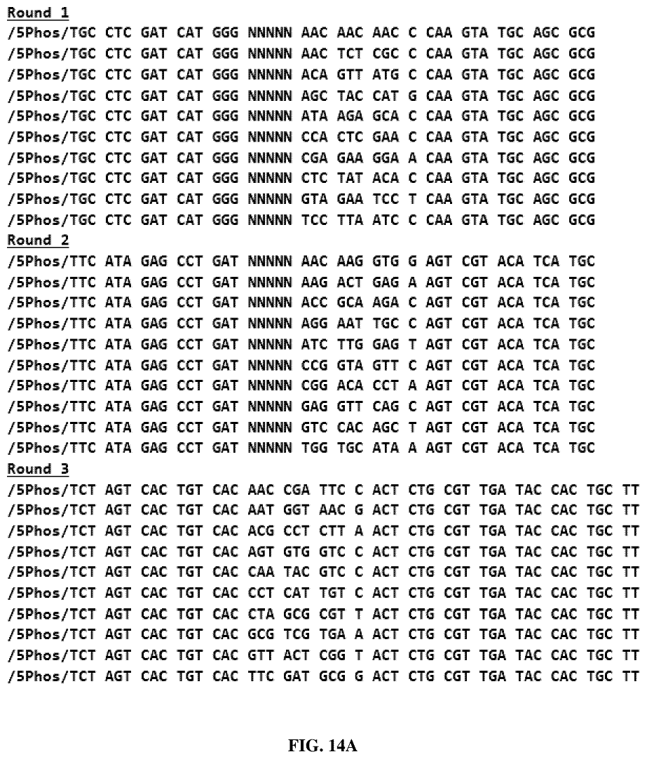

To perform the split-and-pool method, a double label azidolysine peptide was designed and conjugated to oligonucleotides (FIG. 11). To obtain a control (non-split-and-pool) labeling and ligation method to demonstrate that the peptides can be labeled with oligonucleotides and barcoded, the double labeled peptide was isolated by PAGE (FIG. 12C). The presence of peptide was confirmed using a streptavidin shift assay (FIG. 12D). Three rounds of barcode ligation (barcode designs in FIG. 12B) were performed on the labeled peptide and the product was isolated (FIG. 13A). Individual ligation can be seen in FIG. 13A by aliquoting samples after each ligation. Next, PCR was performed to amplify the ligated barcodes and confirm the sequence (FIG. 13B). This product was Sanger sequenced to confirm all three barcodes were correctly ligated.

In more detail, the ligation protocol comprised pre-annealing each round's linker+barcode, ligation of the pre-annealed Round 1 linker-barcode pair to hexynyl oligo by T4 ligase, incubation with Round 1 blocker, repeating ligation & blocking for rounds 2 & 3, and deactivation of the ligase. Barcode, linker, and blocker designs are in FIG. 12B.

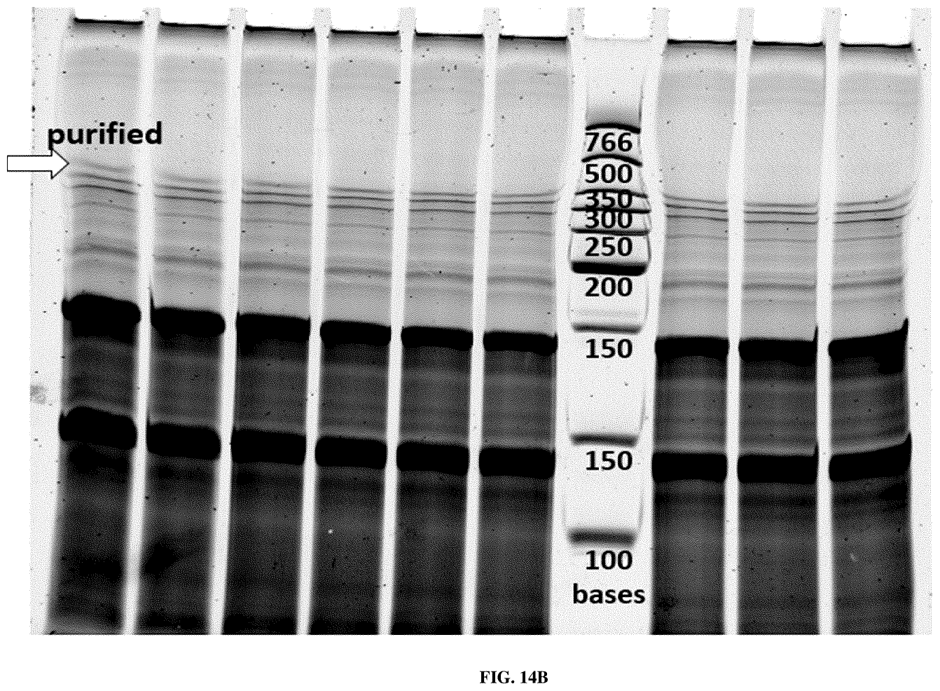

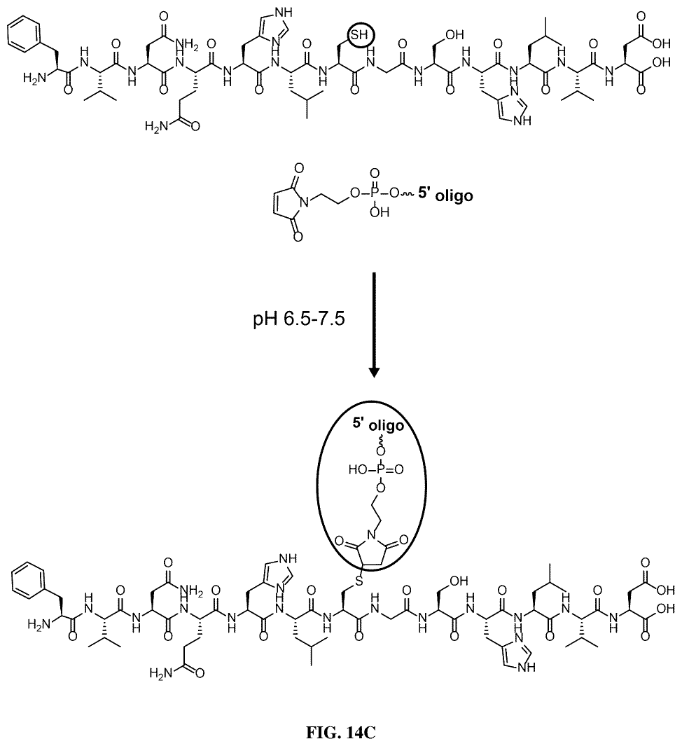

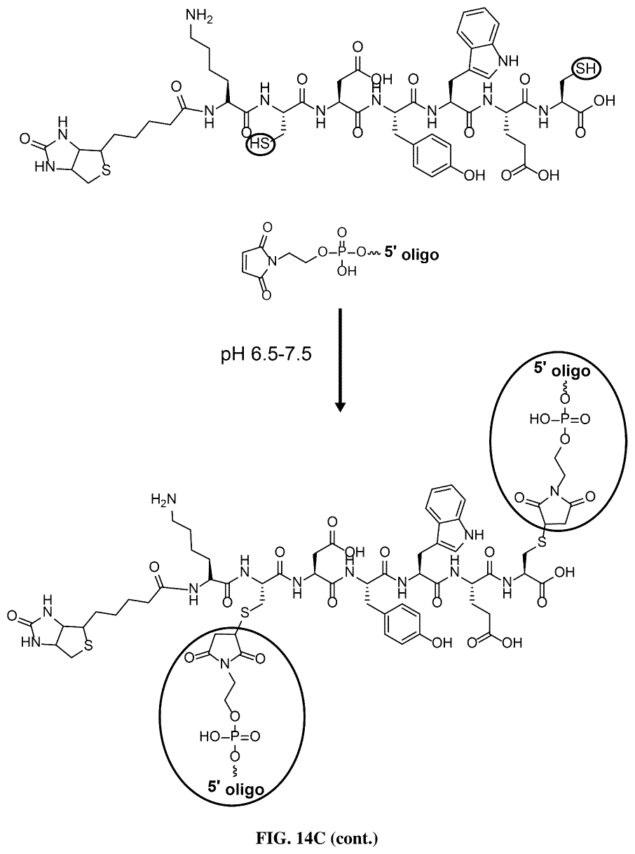

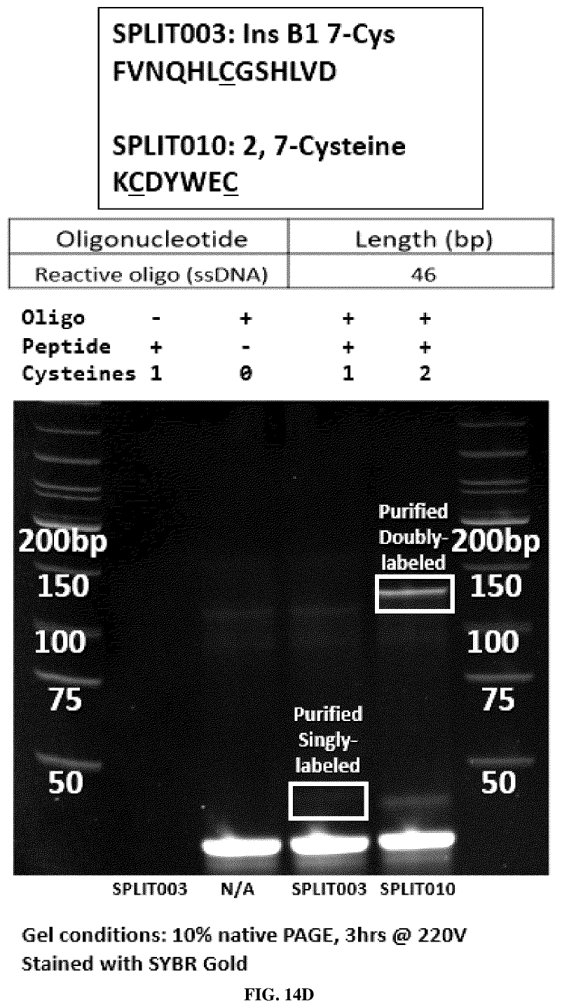

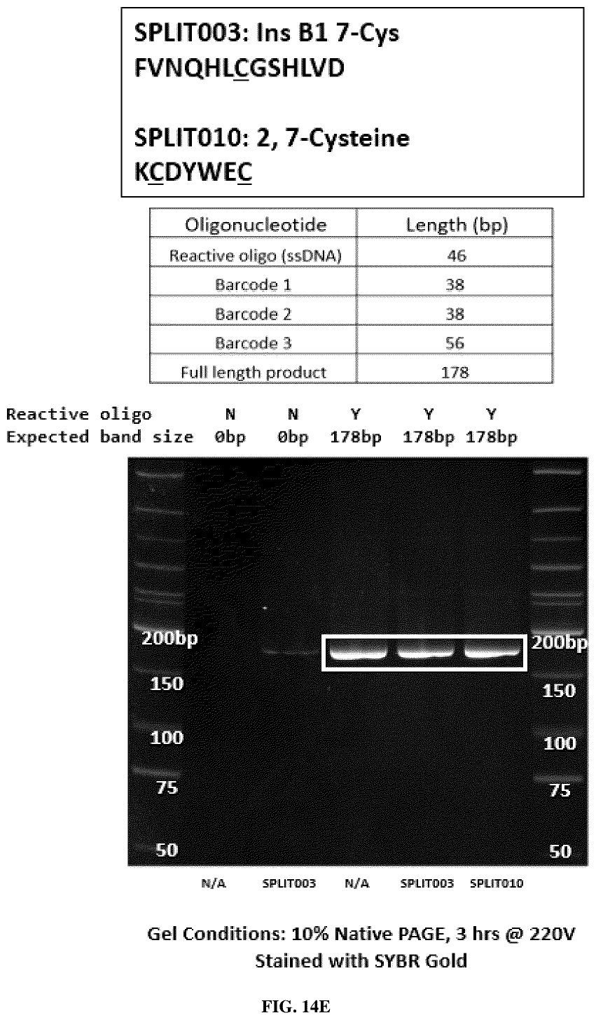

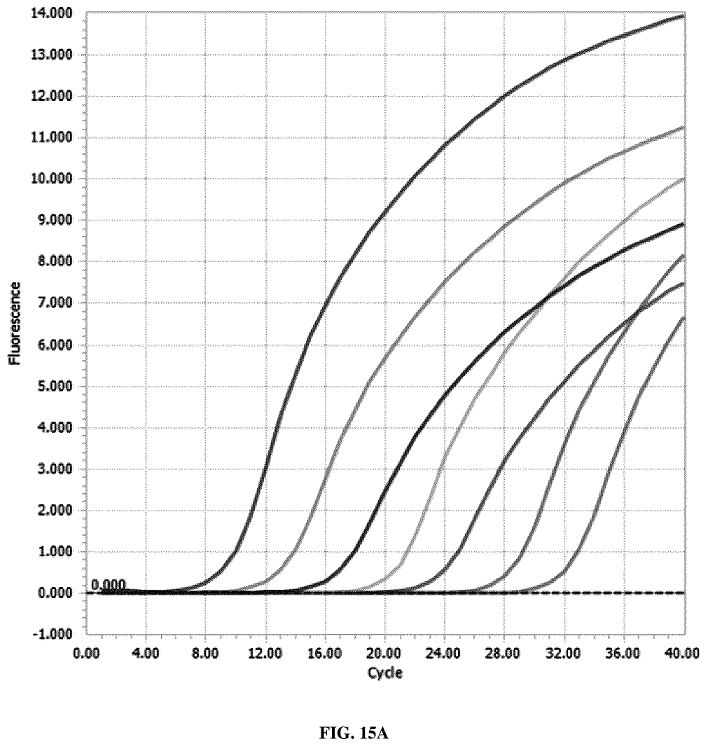

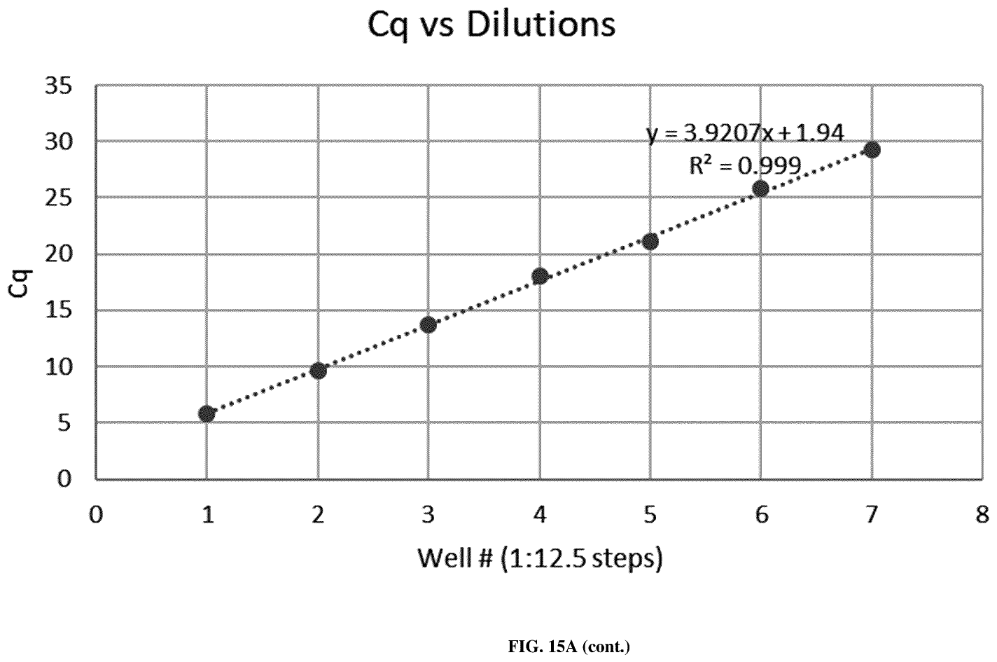

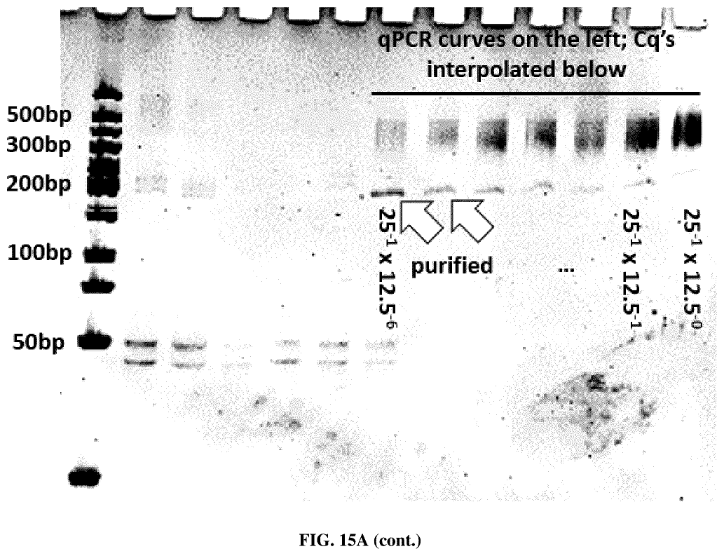

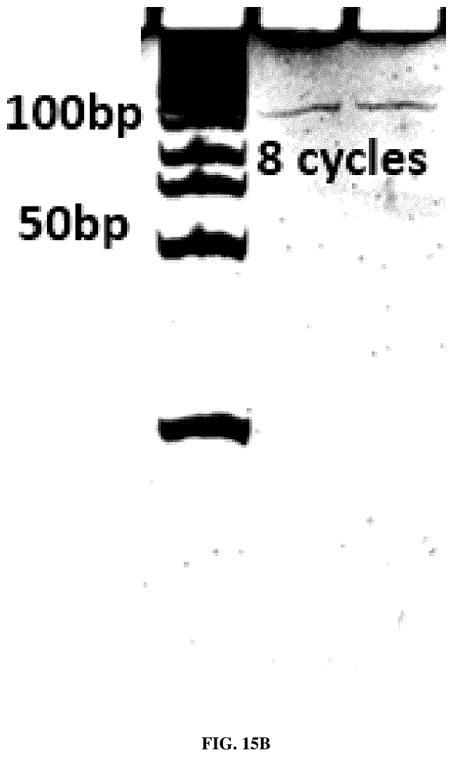

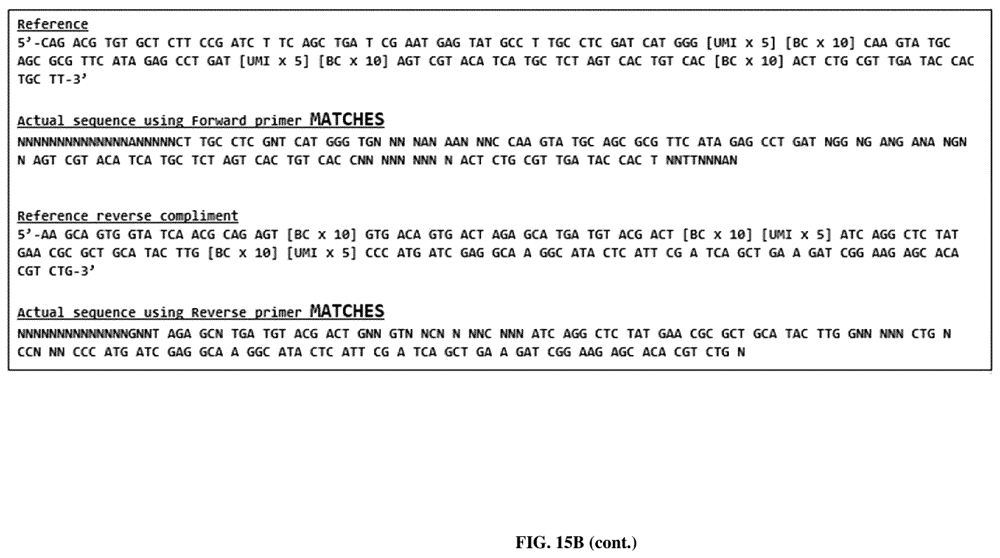

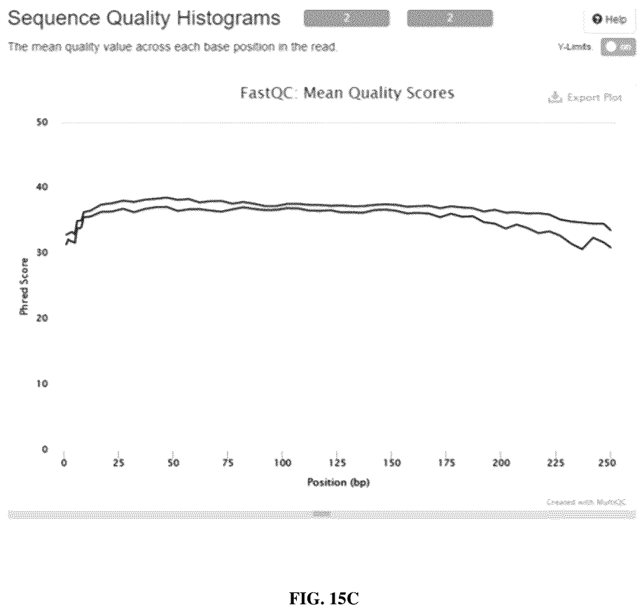

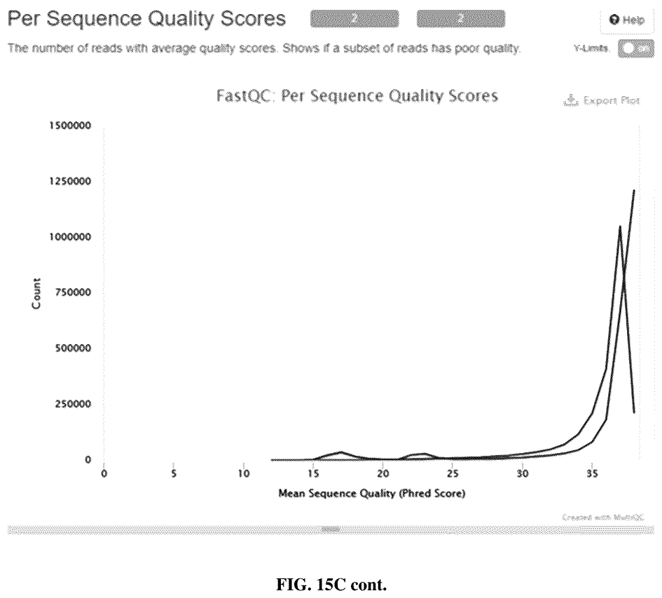

The above experiments were repeated using a split-and-pool of three rounds, with ten barcodes per round. Barcode designs are in FIG. 14A; all linker, blocker, and primer components of the experiment remained identical to FIG. 12B. After three rounds of split-and-pool ligation were performed on purified doubly-labeled peptide and, separately, on hexynyl oligo as a single-label control. Using a doubly-labeled peptide with the 10×10×10 split-pool allows for up to 103=1000 distinct peptides to be identified. Products from both split-and-pool ligations were run on a PAGE gel to confirm size (FIG. 14B) and were PAGE purified. Correct doubly labeled & fully barcoded peptide ˜2×46+2×2×45+2×48=368 bases. The thick band around 150 bases corresponds to the three ligated barcodes not attached to a peptide =45+45+48=138. The top bands were purified. FIG. 15A shows serially diluted qPCR of the purified product. The serial dilution isolates just a few hundred labeled peptides whose barcodes can be sequenced. The linear Cq curve shows the serial dilution was taking place as intended.

FIG. 15B shows amplification & sanger sequencing of the purified product showing the combinatorial barcodes attached to the peptide. The extra N inserted in the barcode/UMI regions of the Sanger sequences is an artifact that is not unexpected. Individual molecules were then sequenceed on an Illumina platform and obtained good reads were obtained, i.e. barcodes were captured from labeled peptides. The sequencing had good quality curves and the vast majority of reads align essentially perfectly to their barcode template.

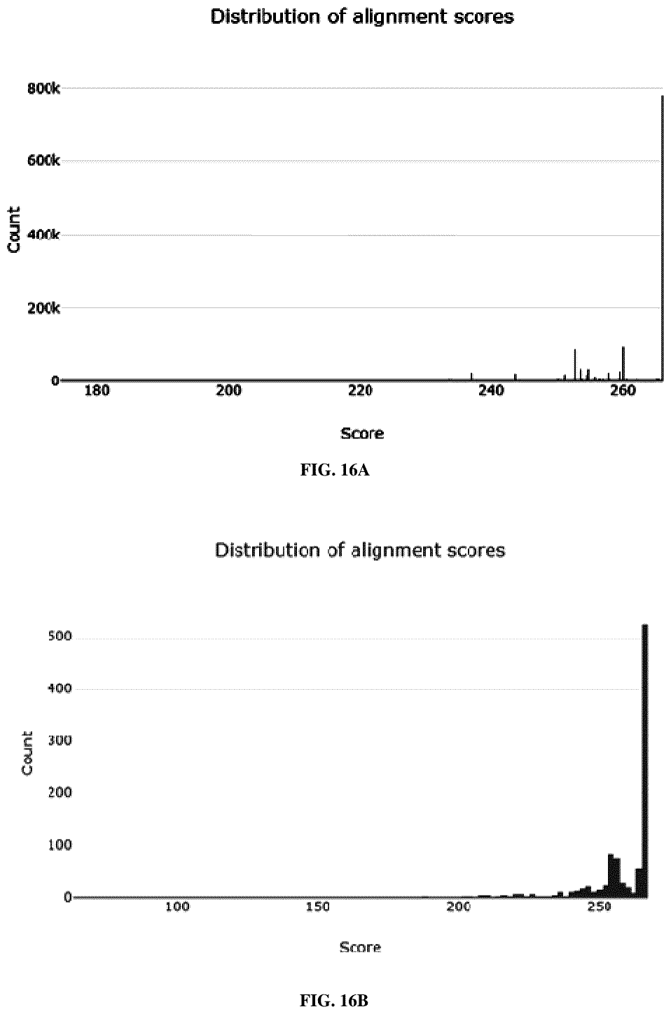

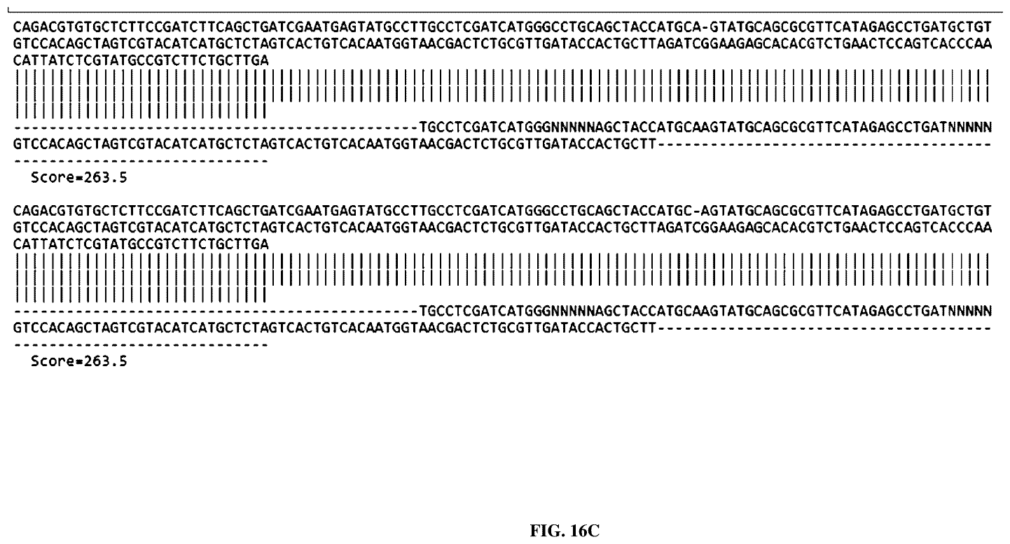

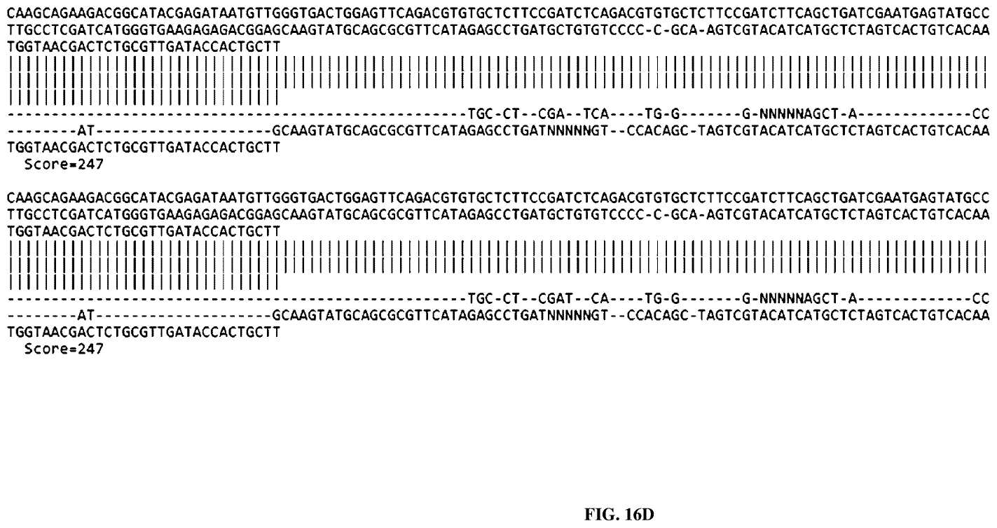

The barcodes were aligned to the reads. The reverse compliment reads were reverse complimented. FIG. 16A shows distribution of the alignment scores with a higher score being better. FIG. 16B shows the best alignment for each combination.

### Example 6

### Cysteine Labeling

It was demonstrated that cysteine-containing peptides can be labeled with maleimide modified oligonucleotides (FIG. 14D). After purifying these products, three rounds of ligation were performed as above (non-split-and-pool) these successful ligations were confirmed via PCR (FIG. 14E).

All of the methods disclosed and claimed herein can be made and executed without undue experimentation in light of the present disclosure. While the compositions and methods of this disclosure have been described in terms of preferred embodiments, it will be apparent that variations may be applied to the methods and in the processes or in the sequence of processes of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide procedural or other details supplementary to those set forth herein, are specifically incorporated herein by reference.
Balister, Handbook of Large-Scale Random Networks (eds. Bollobás, B., Kozma, R. & Miklós, D.) 18, 117-142 (Springer Berlin Heidelberg, 2008).
Bhadra & Ellington, Nucleic Acids Res. 42, e58-e58 (2014).
Bollobás, Random graphs. (Cambridge University Press, 2001).
Buschmann, & Bystrykh, BMC Bioinformatics 14, 272 (2013).
Erdős & Rényi. Acta Math. Acad. Sci. Hung. 12, 261-267 (1964).
Gansner & North, Softw. Pract. Exp. 30, 1203-1233 (2000).
Harary, Graph theory. (Perseus Books, 2001).
Hernandez, New J. Chem. 41, 462-469 (2017).
Kamada & Kawai, Inf. Process. Lett. 31, 7-15 (1989).
Kingman, Poisson processes. (Clarendon Press; Oxford University Press, 1993).
Margulies et al. Nature, 437, 376-380 (2005).
Penrose, Random geometric graphs. (Oxford University Press, 2003).
Pirrung, Angew. Chem. Int. Ed. 41, 1276-1289 (2002).
Schaus, Nat. Commun. 8, (2017).
Shendure, et al. Nature 550, 345-353 (2017).
Söderberg, et al. Nat. Methods 3, 995-1000 (2006).
Soni and Meller, Clin Chem 53:1996-2001 (2007).
Swaminathan, PLOS Comput. Biol. 11, e1004080 (2015).

1. A method for iterative proximity ligation (IPL) comprising:
(a) attaching at least two oligonucleotide tags to at least one molecule of a sample, wherein each oligonucleotide tag comprises (1) a functional group for attachment to said at least one molecule, (2) a primer site, (3) a unique barcode, and (4) a 5′ cleavage half-site or a 3′ cleavage half-site, thereby generating at least one oligonucleotide tag with a 5′ cleavage half-site and at least one oligonucleotide tag with a 3′ cleavage half-site;
(b) ligating said at least one oligonucleotide tag with a 5′ cleavage half-site to said at least one oligonucleotide tag with a 3′ cleavage half-site, thereby generating one or more barcode pairs;
(c) extending the primer in at least one of the oligonucleotide tags of the one or more barcode pairs to generate duplicates of the barcode pairs; and
(d) adding a catalyst to cleave the one or more barcode pairs, thereby reforming said at least two oligonucleotide tags.

2.-41. (canceled)

42. A composition comprising at least two oligonucleotide tags, wherein the first oligonucleotide tag comprises (1) a functional group for attachment to a molecule, (2) a primer site, (3) a unique barcode, and (4) a 5′ restriction half-site ligated to a second oligonucleotide tag, wherein the second oligonucleotide tag comprising comprises (1) a functional group for attachment to a molecule, (2) a primer site, (3) a unique barcode, and (4) a 3′ restriction half-site.

43. The composition of claim 42, wherein each of the oligonucleotides tags are attached to a molecule.

44. A method of determining the spatial position of one or more individual molecules comprising:
(a) obtaining a sample comprising a plurality of individual molecules;
(b) applying multiple rounds of IPL according to claim 1 on said sample to generate a plurality of duplicate barcode pairs;
(c) performing next-generation sequencing on the plurality of duplicate barcode pairs; and
(d) applying pairwise proximity to determine the spatial position of the one or more individual molecules.

45. The method of claim 44, wherein the sample is a biological sample.

46.-47. (canceled)

48. The method of claim 44, wherein the one or more individual molecules are not bound to a solid support.

49. The method of claim 44, wherein each individual molecule is a protein, protein complex, peptide, antibody, carbohydrate, nucleic acid, cell, signaling domain, or a receptor.

50. (canceled)

51. The method of claim 44, wherein each individual molecule is a protein or peptide.

52.-53 (canceled)

54. The method of claim 51, wherein one or more amino acids in the protein or peptide are labeled with amino-acid specific DNA oligonucleotide tags each comprising a unique barcode.

55. The method of claim 54, wherein the labeled amino acids are lysine, cysteine, glutamic acid, aspartic acid, tyrosine, tryptophan, histidine, or any combination thereof.

56. The method of claim 54, wherein the one or more amino acids comprise post-translationally modified side chains.

57. The method of claim 56, wherein the post-translationally modified side chains comprise phosphorylation, glycosylation, methylation, citrullination, or any combination thereof.

58. The method of claim 51, wherein the method further comprises determining the identity and quantity of the protein or peptide.

59. (canceled)

60. The method of claim 44, wherein (b) is performed in a single reaction tube.

61. (canceled)

62. The method of claim 44, wherein the sample is diluted before sequencing in (c).

63. The method of claim 62, wherein each individual molecule is a protein and further comprising digesting the protein with an enzyme.

64.-66. (canceled)

67. The method of claim 63, further comprising applying a second set of IPL rounds to the digested protein.

68. The method of claim 67, further comprising reconciling the duplicate barcode pairs from each set of IPL rounds to identify the protein.

69. The method of claim 44, wherein the one or more individual molecules are nanobeads.

70. The method of claim 69, wherein the method further comprises determining the shape, size, or combination thereof of the nanobeads.

71.-125. (canceled)
